(19) United States
(12) Patent Application Publication
(10) Pub. No.: US 2008/0200437 A1
(43) Pub. Date: Aug. 21, 2008

(54) Cyclitols and Their Derivatives and Their Therapeutic Applications

(76) Inventors: Jean-Marie Lehn, Strasbourg (FR); Srinivasu Pothukanuri, Strasbourg (FR); Alexandros Koumbis, Strasbourg (FR); Carolina Duarte, Strasbourg (FR); Claude Nicolau, Newton, MA (US)

Correspondence Address: KING & SPALDING LLP, 1180 PEACHTREE STREET, ATLANTA, GA, 30309-3521, US

(21) Appl. No.: 11/967,424

(22) Filed: Dec. 31, 2007

Related U.S. Application Data
(60) Provisional application No. 60/877,976, filed on Dec. 29, 2006.

(51) Int. Cl. A61K 31/6615; C07F 9/145; A61P 35/00; A61P 7/00

(52) U.S. Cl. 514/103; 568/14

(57) ABSTRACT

The present invention is directed to polyphosphorylated and pyrophosphate derivatives of cyclitols. More particularly, the invention relates to polyphosphorylated and pyrophosphate derivatives of inositols. The invention also relates to compositions of the polyphosphorylated and pyrophosphate derivatives of inositol and other similar, more lipophilic derivatives, and their use as allosteric effectors, cell-signaling molecule analogs, and therapeutic agents.

### CROSS-REFERENCE TO RELATED APPLICATIONS

This application claims the benefit of U.S. Provisional Patent Application No. 60/877,976 filed Dec. 29, 2006, the contents of which are incorporated herein by reference.

### FIELD OF THE INVENTION

The present invention is directed to polyphosphorylated and pyrophosphate derivatives of cyclitols. More particularly, the invention relates to polyphosphorylated and pyrophosphate derivatives of inositols. The present invention also relates to compositions of the polyphosphorylated and pyrophosphate derivatives of inositol and other similar, more lipophilic derivatives, and their use as allosteric effectors, cell-signaling molecule analogs, and therapeutic agents.

### BACKGROUND OF THE INVENTION

Cyclitols in general, and inositols in particular, exhibit a wide distribution in biological systems, suggesting their importance in biological functions. As a class, cyclitols encompass all polyhydroxylated isocyclic molecules. Inositols refer specifically to the polyhydroxylated cyclohexane derivatives. Inositol has a number of known conformational isomers (i.e. cis-inositol, epi-inositol, allo-inositol, myo-inositol, muco-inositol, neo-inositol, scyllo-inositol, and chiro-inositol), with myo-inositol being the most naturally abundant and well characterized of the conformational isomers. Some polyphosphorylated and pyrophosphate derivatives of inositols are known to possess biological activity. This activity spans from functioning as key secondary messengers in important cell-signaling pathways to the ability to function as allosteric effectors of hemoglobin.

For instance, inositol 1,4,5-trisphosphate is a soluble secondary messenger responsible for the generation of highly organized Ca²⁺ signals in a variety of cell types. These Ca²⁺ signals are known to function in the control of many cellular responses, including cell growth, fertilization, smooth muscle contraction and secretion (1). In addition, inositol 1,3,4,5tetrakisphosphate has been shown to mobilize Ca²⁺ from internal stores through interactions with the inositol 1,4,5trisphosphate receptor (2), and studies have implicated inositol 1,3,4,5tetrakisphospohate in the regulation of Ca²⁺ influx across the plasma membrane (3-8, 29). Inositol 1,4bisphosphate has been reported to exert allosteric activation of muscle-type 6-phosphofructo-1-kinase (9). It has been show that inositol 4,5bisphosphate and inositol 1,4,5trisphosphate, but not inositol 1,3,4,5tetrakisphosphate selectively inhibit Ca²⁺-ATPase of rat heart sarcolemma (10) and of human erythrocyte membrane (11). Inositol 1,3,4,6tetrakisphosphate-activated Ca²⁺ mobilization has been observed in microinjected Xenopus oocytes (12) and in permeablized human neuroblastoma cells (13).

Further, inositol hexaphosphate, including its trispyrophosphate derivatives, have been shown to function as allosteric effectors of hemoglobin (Nicolau et al. U.S. Pat. No. 7,084,115). Hemoglobin is a tetrameric protein which delivers oxygen via an allosteric mechanism. In blood, hemoglobin is in equilibrium between two allosteric structures. In the “T” (for tense) state, hemoglobin is deoxygenated. In the “R” (for relaxed) state, hemoglobin is oxygenated. An oxygen equilibrium curve can be scanned to observe the affinity and degree of cooperatively (allosteric action) of hemoglobin. In the scan, the Y-axis plots the percent of hemoglobin oxygenation and the X-axis plots the partial pressure of oxygen in millimeters of mercury (mmHg). If a horizontal line is drawn from the 50% oxygen saturation point to the scanned curve and a vertical line is drawn from the intersection point of the horizontal line with the curve to the partial pressure X-axis, a value commonly known as P₅₀ is determined (i.e. this is the pressure in mmHg when the scanned hemoglobin sample is 50% saturated with oxygen). Under physiological conditions (i.e. 37° C., pH=7.4, and partial carbon dioxide pressure of 40 mm Hg), the P₅₀ value for normal adult hemoglobin (HbA) is around 26.5 mmHg. If a lower than normal P₅₀ value is obtained for the hemoglobin being tested, the scanned curve is considered to be “left-shifted” and the presence of high-oxygen affinity hemoglobin is indicated. Conversely, if a higher than normal P₅₀ value is obtained for the hemoglobin being tested, the scanned curve is considered to be “right-shifted,” indicating the presence of low oxygen-affinity hemoglobin.

The oxygen release capacity of mammalian red blood cells can be enhanced by introducing allosteric effectors like inositol hexakisphosphate and inositol trispyrophosphate, thereby decreasing the affinity of hemoglobin for oxygen and improving the oxygen economy of the blood. This phenomenon suggests various medical applications for treating individuals suffering from hypoxia related diseases or other conditions associated with inadequate function of the lungs or circulatory system.

For instance, the role of VEGF in the regulation of angiogenesis has been the object of intense investigation (14-19). Whereas VEGF represents a critical, rate-limiting step in physiological angiogenesis, it is also important in pathological angiogenesis, such as that associated with tumor growth (20). VEGF also is known as vascular permeability factor, based on its ability to induce vascular leakage (21) Several solid tumors produce ample amounts of VEGF, which stimulates proliferation and migration of endothelial cells, thereby inducing neovascularization (21). VEGF expression has been shown to significantly affect the prognosis of different kinds of human cancer. Oxygen tension in the tumor has a key role in regulating the expression of the VEGF gene. VEGF mRNA expression is induced by exposure to low oxygen tension under a variety of pathophysiological circumstances (21). Growing tumors are characterized by hypoxia, which induces expression of VEGF also and may be a predictive factor for the occurrence of metastatic disease. Therefore, the ability to increase the oxygen tension in tumor may help inhibit angiogenesis and growth of the tumor. Similar applications also can be envisioned for other angiogenesis related diseases such as hemangioma, rheumatoid arthritis, ulcerative colitis and Crohn's disease.

In addition, it is known that medial temporal oxygen metabolism is markedly affected in patients with mild-to-moderate Alzheimer's disease. It also is known that mean oxygen metabolism in the medial temporal, as well as in the parietal and lateral temporal cortices, is significantly lower in patients with Alzheimer's disease than in control groups without Alzheimer's disease (22). Thus, one potential means of treating patients with Alzheimer's disease is to increase oxygen across the blood brain barrier using an allosteric effector.

Allosteric effectors also may help in the treatment of a variety of diseases associated with various forms of dementia. Because the brain relies on a network of vessels to bring it oxygen-bearing blood, if the oxygen supply to the brain fails, brain cells are likely to die which can cause symptoms of vascular dementia. These symptoms can occur either suddenly following a stroke, or over time though a series of small strokes. Thus, one potential means for treating patients with vascular diseases associated with various forms of dementia is to increase the oxygen available to affected areas such as across the blood brain barrier.

Moreover, treatment of an individual with an allosteric effector may have beneficial effects for both the treatment of stroke and the condition of osteoporosis that can sometime follow. Although, stroke and the bone-thinning disease, osteoporosis, are usually thought of as two distinct health problems, it has been found there is a connection between the two. Patients who survive strokes are significantly more likely to suffer from osteoporosis, a disease that puts them at high risk for bone fractures. Often the fractures occur on the side of the body that has been paralyzed from the stroke. It is known that a stroke occurs when the supply of blood and oxygen to the brain ceases or is greatly reduced. If a portion of the brain loses its supply of nutrient-rich blood and oxygen, the bodily functions controlled by that part of the brain (vision, speaking, walking, etc.) are impaired. Annually, more than 500,000 people in the United States suffer strokes and 150,000 die as a result thereof. One means of increasing oxygen flow to the brain is by using of an allosteric effector of hemoglobin.

Therefore, the ability to readily synthesize polyphosphorylated and pyrophosphate derivatives of cyclitols will be a valuable tool for uncovering new allosteric effectors suitable for the potential therapeutic uses mentioned above. In addition, given the diversity of cell types and cell functions that rely on Ca²⁺ signaling and the role of cyclitols in conducting those signals, the ability to readily synthesize polyphosphate and pyrophosphate derivatives, will provide an invaluable tool in better elucidating the function of these complex signaling pathways. It also will be useful for determining any therapeutic activity these derivatives may have including the ability to function as prodrugs. The biological activity of myo-inositol has been fairly well characterized. However, there are a number of conformational isomers of inositol of which biological functions are either not known or are poorly understood. Therefore, the ability to readily synthesize polyphosphorylated and pyrophosphate derivatives of these conformational isomers of inositol also will potentially unlock a number of useful and heretofore unknown biological activities.

### SUMMARY OF THE INVENTION

The present invention is directed to compounds and compositions comprising polyphosphorylated and pyrophosphate derivatives of cyclitols, in particular inositols, and methods for their synthesis. In addition, the present invention is directed to the use of these compositions as allosteric effectors of hemoglobin, cell-signaling molecule analogs and as therapeutic agents in treating diseases caused by hypoxia or other conditions associated with inadequate function of the lungs or circulatory system.

In one embodiment, the present invention is a compound that is a hexakisphophate derivative of inositol. More specifically, the triethylammonium salts of hexakisphosphate derivatives of cis-inositol, epi-inositol, allo-inositol, muco-inositol, neo-inositol, scyllo-inositol, (+) chiro-inositol, or (−) chiro-inositol In another embodiment, the compound is a polyphosphorylated inositol derivative containing one or more free hydroxyl or hydroxyl derivative groups, such as an alkoxy and acyloxy groups.

In another embodiment, the present invention is a compound that is a pyrophosphate derivative of inositol. The inositol derivative may be a monopyrophosphate, bispyrophosphate, or trispyrophosphate derivative. In another embodiment, the compounds are trisphosphorimide derivatives or tristhiopyrophosphate derivatives of inositol.

In another embodiment, the present invention comprises the corresponding salts of the polyphosphorylated and pyrophosphate derivatives of inositol. The salt complex may be formed with an alkali metal cation, alkaline metal cation, ammonium cation, or organic cation.

In another embodiment, the present invention comprises pharmaceutical compositions comprising the polyphosphorylated and/or pyrophosphate derivatives of inositol.

In yet another embodiment, the present invention is directed to the use of polyphosphorylated and pyrophosphate inositols in a method of reducing the affinity of hemoglobin for the blood.

In another embodiment the compounds and compositions of the present invention are used as therapeutic agents for treating disease caused by hypoxia or other conditions associated with inadequate function of the lungs or circulatory system.

In another embodiment of the invention, the compounds and compositions of the present invention may be used as analogs of naturally occurring inositol cell signaling compounds or prodrugs thereof.

### BRIEF DESCRIPTION OF THE DRAWINGS

FIG. 1 depicts the different conformational isomers of inositol.

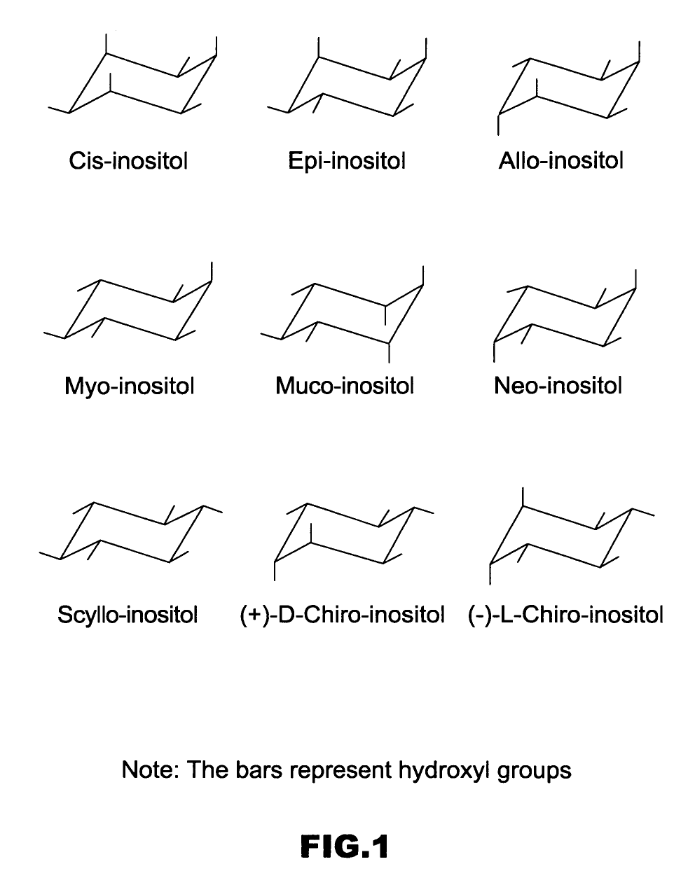

FIG. 2 depicts known and suggested pathways of inositol metabolism.

### DETAILED DESCRIPTION OF THE INVENTION

The present invention is directed to polyphosphorylated and pyrophosphate derivatives of cyclitols, in particular inositols. Methods for synthesizing the compounds of the present invention are described below. The present invention also encompasses the use of the polyphosphorylated and pyrophosphate derivatives of cyclitols as allosteric effectors of hemoglobin. In addition, the present invention encompasses their use as therapeutic agents for treatment of hypoxia-related diseases or other conditions associated with inadequate function of the lungs or circulatory system. The present invention also encompasses the use of polyphosphorylated and pyrophosphate derivates as useful intermediates in studying cell-signaling pathways or the design of new therapeutic agents for modulating such pathways, in particular those cell-signaling pathways that transmit signals through cleavage of phosphoinositol lipids.

### Definitions

For convenience, certain terms employed in the specification, examples, and appended claims are collected here. As used throughout this specification and claims, the following terms have the following meanings:

The term “hemoglobin” includes all naturally- and non-naturally-occurring hemoglobin.

The term “hemoglobin preparation” includes hemoglobin in a physiologically compatible carrier or lyophilized hemoglobin reconstituted with a physiologically compatible carrier, but does not include whole blood, red blood cells or packed red blood cells.

The term “whole blood” refers to blood containing all its natural constituents, components, or elements or a substantial amount of the natural constituents, components, or elements. For example, it is envisioned that some components may be removed by the purification process before administering the blood to a subject.

“Purified,” “purification process,” and “purify” all refer to a state or process of removing one or more compounds of the present invention from the red blood cells or whole blood such that when administered to a subject the red blood cells or whole blood is nontoxic.

“Non-naturally-occurring hemoglobin” includes synthetic hemoglobin having an amino-acid sequence different from the amino-acid sequence of hemoglobin naturally existing within a cell, and chemically-modified hemoglobin. Such non-naturally-occurring mutant hemoglobin is not limited by its method of preparation, but is typically produced using one or more of several techniques well known in the art, including, for example, recombinant DNA technology, transgenic DNA technology, protein synthesis, and other mutation-inducing methods.

“Chemically-modified hemoglobin” is a natural or non-natural hemoglobin molecule which is bonded to another chemical moiety. For example, a hemoglobin molecule can be bonded to pyridoxal-5′-phosphate, or other oxygen-affinity-modifying moiety to change the oxygen-binding characteristics of the hemoglobin molecule, to crosslinking agents to form crosslinked or polymerized hemoglobin, or to conjugating agents to form conjugated hemoglobin.

“Oxygen affinity” means the strength of binding oxygen to a hemoglobin molecule. High oxygen affinity means hemoglobin does not readily release its bound oxygen molecules. The P₅₀ is a measure of oxygen affinity.

“Cooperativity” refers to the sigmoidal oxygen-binding curve of hemoglobin, i.e. the binding of the first oxygen to one subunit within the tetrameric hemoglobin molecule enhances the binding of oxygen molecules to other unligated subunits. It is conveniently measured by the Hill coefficient (n[max]). For Hb A, n[max]=3.0.

The term “treatment” is intended to encompass also prophylaxis, therapy and cure.

“Ischemia” means a temporary or prolonged lack or reduction of oxygen supply to an organ or skeletal tissue. Ischemia can be induced when an organ is transplanted, or by conditions such as septic shock and sickle cell anemia.

“Skeletal tissue” means the substance of an organic body of a skeletal organism consisting of cells and intercellular material, including but not limited to epithelium, the connective tissues (including blood, bone and cartilage), muscle tissue, and nerve tissue.

“Ischemic insult” means damage to an organ or skeletal tissue caused by ischemia.

“Subject” means any living organism, including human, and animals.

The phrases “parenteral administration’ and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrastemal injection and infusion.

As used herein, the term “surgery” refers to the treatment of diseases, injuries, and deformities by manual or operative methods. Common surgical procedures include, but are not limited to, abdominal, aural, bench, cardiac, cineplastic, conservative, cosmetic, cytoreductive, dental, dentofacial, general, major, minor, Moh's, open heart, organ transplantation, orthopedic, plastic, psychiatric, radical, reconstructive, sonic, stereotactic, structural, thoracic, and veterinary surgery. The method of the present invention is suitable for patients that are to undergo any type of surgery dealing with any portion of the body, including, but not limited to, those described above, as well as any type of any general, major, minor, or minimally invasive surgery.

“Minimally invasive surgery” involves puncture or incision of the skin, or insertion of an instrument or foreign material into the body. Non-limiting examples of minimal invasive surgery include arterial or venous catheterization, transurethral resection, endoscopy (e.g. laparoscopy, bronchoscopy, uroscopy, pharyngoscopy, cystoscopy, hysteroscopy, gastroscopy, coloscopy, colposcopy, colioscopy, sigmoidoscopy, and orthoscopy), and angioplasty (e.g., balloon angioplasty, laser angioplasty, and percutaneous transluminal angioplasty).

The term “ED₅₀” means the dose of a drug that produces 50% of its maximum response or effect. Alternatively, the dose that produces a pre-determined response in 50% of test subjects or preparations.

The term “LD₅₀” means the dose of a drug that is lethal in 50% of test subjects.

The term “therapeutic index” refers to the therapeutic index of a drug defined as LD₅₀/ED₅₀.

The phrases “systemic administration,” “administered systemically,” “peripheral administration,” and “administered peripherally” as used herein mean the administration of a compound, drug or other material other than directly into the central nervous system, such that it enters the patient's system, and thus, is subject to metabolism and other like processes, for example, subcutaneous administration.

The term “structure-activity relationship (SAR)” refers to the way in which altering the molecular structure of drugs alters their interaction with a receptor, enzyme, etc.

The term “pyrophosphate” refers to the general formula below:

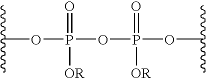

wherein R is selected independently for each occurrence from the group consisting of H, cations and hydrocarbon groups.

The terms “internal pyrophosphate moiety,” “internal pyrophosphate ring,” and “cyclic pyrophosphate” refer to the structure feature below:

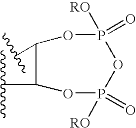

wherein R is selected independently for each occurrence from the group consisting of H, cations, alkyl, alkenyl, alkynyl, aralkyl, aryl, and acyl groups.

The term “IHP-monopyrophosphate” (abbreviated as “IMPP”) refers to inositol hexakisphosphate where two orthopyrophosphates are condensed to one internal pyrophosphate ring.

The term “IHP-trispyrophosphate” or “inositol trispyrophosphate” (both abbreviated as “ITPP”) refers to inositol hexakisphosphate with three internal pyrophosphate rings.

The term “2,3-diphosph-D-glyceric acid” (DPG) refers to the compound below:

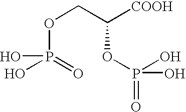

The term “2,3-cyclopyrophosphoglycerate” (CPPG) refers to the compound below:

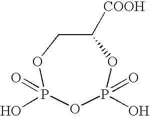

The term “ammonium cation” refers to the structure below:

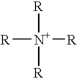

wherein R represents independently for each occurrence H or a substituted or unsubstituted aliphatic group. An “aliphatic ammonium cation” refers to the above structure when at least one R is an aliphatic group. A “quaternary ammonium cation” refers to the above structure when all four occurrences of R independently represent aliphatic groups. R can be the same for two or more occurrences or different for all four.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e. the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

The term “aralkyl,” as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group as defined above but having from approximately one to approximately ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure also may be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃. —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, of which ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, intro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle,” as used herein, refers to an aromatic or non-aromatic ring in which each atom of the ring is carbon.

As used herein, the term “nitro” means —NO₂ the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

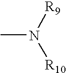

wherein R₉, R₁₀ and R′₁₀ each independently represent a hydrogen, an alkyl, an alkenyl, —(CH2)ₘ—R₈, or R₉ and R₁₀ taken together with the N atom to which they are attached complete a heterocycle having from 4 to 5 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH2)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

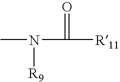

wherein R₉ is as defined above, and R′₁₁ represents a hydrogen, an alkyl, an alkenyl or —(CH2)ₘ—R₈, where m and R₈ are as defined above.

The term “amido” is art recognized as an amino-substituted carbonyl and includes a moiety that can be represented by the general formula:

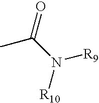

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —(CH2)ₘ—R₈, wherein m and R₈ are defined above Representative alkylthio groups include methylthio, ethyl thio, and the like.

The term “carbonyl” is art recognized and includes such moieties as can be represented by the general formula:

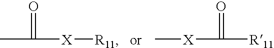

wherein X is a bond or represents an oxygen or a sulfur, and R₁₁ represents a hydrogen, an alkyl, an alkenyl, —(CH2)ₘ—R₈ or a pharmaceutically acceptable salt, R′₁₁ represents a hydrogen, an alkyl, an alkenyl or —(CH2)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R′₁₁ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH2)ₘR₈, where m and R₈ are described above.

The term “sulfonate” is art recognized and includes a moiety that can be represented by the general formula:

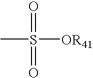

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

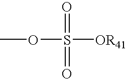

in which R₄₁ is as defined above.

The term “sulfonamido” is art recognized and includes a moiety that can be represented by the general formula:

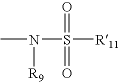

in which R₉ and R′₁₁ are as defined above.

The term “sulfamoyl” is art-recognized and includes a moiety that can be represented by the general formula:

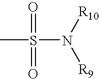

in which R₉ and R₁₀ are as defined above.

The term “sulfonyl”, as used herein, refers to a moiety that can be represented by the general formula:

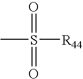

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

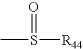

in which R₄₄ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carhonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

A “angiogenesis-related disease” as defined herein includes, but is not limited to, excessive or abnormal stimulation of endothelial cells (e.g. atherosclerosis), blood borne tumors, solid tumors and tumor metastasis, benign tumors, for example, hemangiomas, acoustic neuromas, neurofribromas, trachomas, and pyogenic granulomas, vascular malfunctions, abnormal wound healing, inflammatory and immune disoreders, Bechet's disease, gout, or gouty arthritis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease). Cancers that may be treated by the present invention include, but is not limited to, breast cancer, prostate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple mycloma.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover which is incorporated herein by reference.

### Use as Allosteric Effectors and Therapeutic Agents

The present invention encompasses the use of the polyphosphorylated and pyrophosphate cyclitol derivatives of the present invention as allosteric effectors of hemoglobin and therapeutic agents. In one embodiment the allosteric effector is a polyphosphorylated inositol. In yet another embodiment, the allosteric effector is an inositol pyrophosphate derivative. The process of allosterically modifying hemoglobin towards a low oxygen affinity state can be used in a variety of applications in treatments for ischemia, angiogenesis related diseases, such as cancer, and ischemia mediated diseases such as Alzheimer's disease, dementia, stroke, chronic obstructive pulmonary disease (COPD), osteoporosis, adult respiratory distress syndrome (ARDS), etc., in extending the shelf-life of blood or restoring the oxygen carrying capacity of out-dated blood, and as sensitizers for x-ray irradiation, as well as many other applications.

Because the compounds, compositions, and methods of the present invention may be capable of allosterically modifying hemoglobin to favor the low oxygen affinity “T” state, the compounds of the present invention may be useful in treating a variety of disease states in mammals, including humans, wherein tissues suffer from low oxygen tension, such as cancer, ischemia, Alzheimer's disease, dementia, and stroke. Furthermore, as described by Hirst et al. (23) decreasing the oxygen affinity of hemoglobin in circulating blood has been shown to be beneficial in the radiotherapy of tumors. Compounds of the present invention may also be administered to patients in whom the affinity of hemoglobin for oxygen is abnormally high. For example, certain hemoglobinopathies, certain respiratory distress syndromes, e.g. respiratory distress syndromes of new born infants aggravated by high fetal hemoglobin levels, and conditions in which the availability of hemoglobin/oxygen to the tissues is decreased (e.g., in ischemic conditions such as peripheral vascular disease, coronary occlusion, congestive heart failure, cerebral vascular accidents, or tissue transplant). The compounds and compositions may also be used to inhibit platelet aggregation, antithrombotic purposes, and wound healing.

Additionally, the compounds and compositions of the present invention may be added to whole blood or packed cells preferably at the time of storage or at the time of transfusion to facilitate the dissociation of oxygen from hemoglobin and improve the oxygen delivering capability of the blood. When blood is stored, the hemoglobin in the blood tends to increase its affinity for the oxygen losing 2,3-diphosphoglycerides. As described above, the compounds and compositions of the present invention is capable of reversing and/or preventing the functional abnormality of hemoglobin observed when whole blood or packed cells are stored. The compounds and compositions can added to whole blood or red blood cell fractions in a closed system using an appropriate reservoir in which the compound or composition is placed prior to storage or which is present in the anticoagulating solution in the blood collecting bag.

The compounds, compositions and methods of this invention can be used to cause more oxygen to be delivered at low blood flow and low temperatures, providing the ability to decrease or prevent the cellular damage, e.g., mycocardial or neuronal, typically associated with hypoxic conditions.

The compounds, composition and methods of this invention can be used to decrease the number of red blood cells required for treating hemorrhagic shock by increasing the efficiency with which they deliver oxygen.

Damaged tissues heal faster when there is better blood flow and increased oxygen tension. Therefore, the compounds, compositions and methods of this invention can be used to speed wound healing. Furthermore, by increasing the oxygen delivery to wounded tissue, the compounds, compositions and methods of this invention can play a role in the destruction of infection causing bacteria at a wound.

The compounds, compositions, and methods of the present invention may be effective in enhancing the delivery of oxygen to the brain, especially before complete occlusion and reperfusion injuries occur due to free radical formation such as those that might occur after stroke. In addition, it is known that medial temporal oxygen metabolism is markedly affected in patients with mild-to-moderate Alzheimer's disease. It is also known that mean oxygen metabolism in the medial temporal, as well as in the parietal and lateral temporal cortices is significantly lower in patients with Alzheimer's disease than in control groups without Alzheimer's disease (22). Thus one means of treating patients with Alzheimer's disease is to increase oxygen across the blood brain barrier using an allosteric effector according to the present invention.

The compounds, compositions and methods of the present invention are capable of increasing oxygen delivery to blocked arteries and surrounding muscle and tissues, thereby relieving the distress of angina attacks.

Acute respiratory disease syndrome (ARDS) is characterized by interstitial and/or alveolar damage and hemorrhage as well as perivascular lung edema associated with the hyaline membrane, proliferation of collagen fibers, and swollen epithelium with increased pinocytosis. The enhanced oxygen delivering capacity that is provided to RBCs by the compounds, compositions and methods of this invention can be used in the treatment and prevention of ARDS by mitigating against lower than normal oxygen delivery to the lungs.

There are several aspects of cardiac bypass surgery that make attractive the use of compounds or compositions or method of the present invention. First, the compounds and compositions of the present invention can act as neuroprotective agents. After cardiac bypass surgery, up to 50% of patients show some signs of cerebral ischemia based on tests of cognitive function. Up to 5% of these patients show evidence of stroke. Second, cardioplegia is the process of stopping the heart and protecting the heart from ischemia during heart surgery. Cardioplegia is performed by perfusing the coronary vessels with solutions of potassium chloride and the bathing the heart in ice water. However, blood cardioplegia is also used. This is where potassium chloride is dissolved in blood instead of salt water. During surgery the heart is deprived of oxygen and the cold temperature helps slow down metabolisms. Periodically during this process, the heart is perfused with the cardioplegia solution to wash out metabolites and reactive species. Cooling the blood increases the oxygen affinity of hemoglobin, thus making oxygen unloading less efficient. However, treatment of blood cardioplegia with RBC's or whole blood previously treated with compounds or compositions of the present invention and subsequently purified can counteract the effects of cold on oxygen affinity and make oxygen release to the ischemic myocardium more efficient, thereby improving cardiac function after the heart begins to beat again. Third, during bypass surgery the patient's blood is diluted for the process of pump prime. This hemodilution is essentially acute anemia. Because the compounds and compositions of the present invention make oxygen transport more efficient, their use during hemodilution (whether in bypass surgery or other surgeries, such as orthopedic or vascular) would enhance oxygenation of the tissues in an otherwise compromised condition. Additionally, the compounds and methods of the present invention also find use in patients undergoing angioplasty, who may experience acute ischemic insult, e.g. due to the dye(s) used in this procedure.

Recently Nicolau et al. (U.S. Application Publication No. 2006/0258626) have demonstrated the ability of inositol tripyrophosphate to decrease VEGF expression. VEGF represents a critical, rate-limiting step in physiological angiogenesis, VEGF is also important in pathological angiogenesis, such as that associated with tumor growth (20). VEGF is also known as vascular permeability factor, based on its ability to induce vascular leakage (21). Several solid tumors produce ample amounts of VEGF, which stimulates proliferation and migration of endothelial cells, thereby inducing neovascularization (21,30). VEGF expression has been shown to significantly affect the prognosis of different kinds of human cancer. Oxygen tension in the tumor has a key role in regulating the expression of the VEGF gene. VEGF mRNA expression is induced by exposure to low oxygen tension under a variety of pathophysiological circumstances (21). Growing tumors are characterized by hypoxia, which induces expression of VEGF and may also be a predictive factor for the occurrence of metastatic disease. Therefore the compounds and compositions of the present invention may also be useful in down-regulating VEGF expression and used in treating angiogenesis related diseases such as cancer.

### Use as Cell-Signaling Analogs

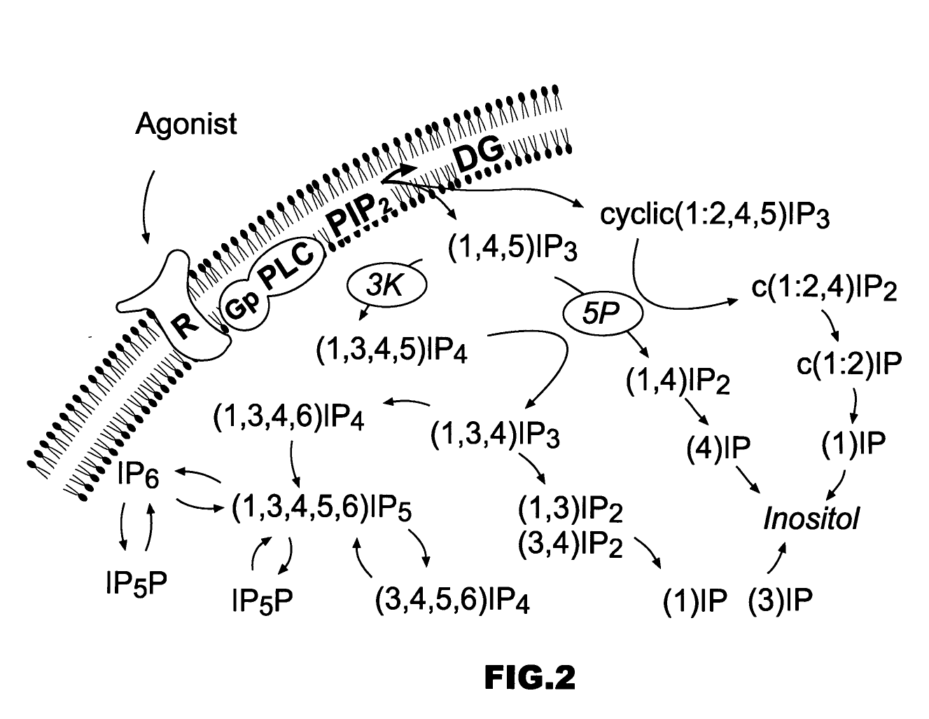

Activation of a variety of cell surface receptors results in the phospholipase C-catalyzed hydrolysis of the minor plasma membrane phospholipid phosphatidylinositol 4,5-bisphosphate, with concomitant formation of inositol 1,4,5-trisphosphate and diacylglycerol (4). It is accepted that inositol 1,4,5-trisphosphate is a crucial second messenger that releases Ca²⁺ from stores associated with the endoplasmic reticulum and that such cytosolic Ca²⁺ signals induce diverse cellular responses, including cell growth and development, fertilization, secretion, smooth muscle contraction, sensory perception, and neuromodulation (24, 25). However, the metabolic pathway, including the kinases, phosphatases and receptors, by which inositol intermediates facilitate this signaling is amazingly complex as shown in FIG. 2. Indeed there is an increasing appreciation that other polyphosphorylated forms of inositol may play a role as crucial intracellular messengers or perhaps a unique role in protein phosphorylation (26, 27). The high affinity of inositol trisphosphate receptors for inositol (1,4,5)-trisphosphate has allowed for the development of a simple radioreceptor assay (28) to quantify inositol trisphosphate mass from cell and tissue extracts. The accessibility of mass assays for this messenger as well as its lipid precursor and its kinase derived product inositol tetrakisphosphate has been invaluable in recent investigations of these intracellular pathways and in the evaluation of the enormous number of GPCRs that use this signaling pathway (24).

In order to determine if inositol receptor specific ligands can be developed or whether cell-permeable inhibitors of the enzymes that metabolize inositol prove to be useful therapeutic agents requires a still better understanding of this signaling pathway and its associated proteins (24). The ability to readily synthesis polyphosphorylated and pyrophosphate inositol derivatives provided by the present invention will be useful in further understanding this signaling pathway and identifying and designing effective therapeutic targets.

### Formulations and Pharmaceutical Compositions

The compounds and compositions described herein can be provided as physiologically acceptable formulations using known techniques, and the formulations can be administered by standard routes. In general, the compositions can be administered by topical, oral, rectal, nasal, inhalation or parenteral (e.g., intravenous, subcutaneous, intramuscular, intradermal, intraocular, intratracheal or epidural) routes. In addition, the compositions can be incorporated into polymers allowing for sustained release, the polymers being implanted in the vicinity of where delivery is desired, for example, at the site of a tumor or within or near the eye, or the polymers can be implanted, for example, subcutaneously or intramuscularly or delivered intravenously or intraperitoneally to result in systemic delivery of the analog of the composition. Other formulations for controlled, prolonged release of therapeutic agents useful in the present invention are disclosed in U.S. Pat. No. 6,706,289.

Formulations contemplated as part of the present invention include nanoparticle formulations made by methods disclosed in U.S. patent application Ser. No. 10/392,403 (Publication No. 2004/0033267). By forming nanoparticles, the compositions disclosed herein are shown to have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations can conveniently be presented in unit dosage form and can be prepared by conventional pharmaceutical techniques. Such techniques include the step of bringing into association the active ingredient(s) and a pharmaceutical carrier(s) or excipient(s). In general, the formulations are prepared by uniformly and intimately bringing into association the active ingredient(s) with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient or ingredients; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound or compounds moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored base, usually sucrose and acacia or tragacanth; pastilles comprising the active ingredients in an inert base such as gelatin and glycerin, or sucrose and acacia; and mouthwashes comprising the ingredient to be administered in a suitable liquid carrier.

Formulations suitable for topical administration to the skin may be presented as ointments, creams, gels or pastes comprising the ingredient to be administered in a pharmaceutical acceptable carrier. In one embodiment the topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers that are known in the art to be appropriate.

Formulations suitable for inhalation may be presented as mists, dusts, powders or spray formulations containing, in addition to the active ingredient, ingredients such as carriers that are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration include particulate preparations of the anti-angiogenic agents, including, but not limited to, low-micron, or nanometer (e.g., less than 2000 nanometers, preferably less than 1000 nanometers, most preferably less than 500 nanometers, especially less than 75 nanometers in average cross section) sized particles, which particles are comprised of 2-methoxyestradiol analogs and/or one or more anti-cancer agents alone or in combination with accessory ingredients or in a polymer for sustained release. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

It should be understood that, in addition to the ingredients particularly mentioned above, the formulations of the present invention may include other agents conventional in the art having regard to the type of formulation in question, for example, those suitable for oral administration may include flavoring agents, and nanoparticle formulations (e.g. less than 2000 nanometers, preferably less than 1000 nanometers, most preferably less than 500 nanometers, especially less than 75 nanometers in average cross section) may include one or more than one excipient chosen to prevent particle agglomeration.

### Compounds of the Present Invention

In one embodiment the polyphosphorylated cyclitol derivatives are polyphosphorylated inositols. The polyphosphorylated inositols may include one or more free hydroxyl groups or hydroxyl derivative groups. The free hydroxyl or hydroxyl derivative groups can be synthesized in a stereoselective or non-stereoselective manner. Polyphosphorylated derivatives of all conformational isomers of inositol are encompassed by this invention.

In another embodiment, the pyrophosphate derivatives of cyclitols are pyrophosphate derivatives of inositols. The pyrophosphate derivatives can be monopyrophosphate, bispyrophosphate, or trispyrophosphate inositols. The cyclitol pyrophosphates of the present invention, in particular the inositol pyrophosphates, may be converted to their corresponding phosphorimides or thiopyrophosphates. Pyrophosphate derivatives of all conformational isomers of inositol are encompassed by this invention.

Schemes 1 through 7 below and the experimental description outline the synthetic methods that may be used to prepare the compounds of the present invention. It is understood the synthetic transformations outlined below can be carried out with a variety of alternate reagents that function to achieve the desired reaction.

### Polyphosphates of Cyclitols

Reaction of cyclitols with phosphorylating agents in the presence of activating agents yields protected polyphosphorylated derivatives, which are thereafter deprotected and the phosphorylated cyclitols obtained are best isolated, purified and conserved as their sodium salts. Other salts, such as ammonium salts, or salts of alkali earth metals, alkaline earth metals, or organic cations, may be prepared and serve a similar purpose.

The synthetic routes for preparing these polyphosphorylated derivatives are described below in the preparation of compounds 10 and 11 of Scheme 2, compounds 8 and 9 of Scheme 4, and compounds 1 and 2 of Scheme 5.

Further, it is possible to prepare selectively phosphorylated derivatives of cyclitols that contain precisely located free hydroxyl groups or derivatives thereof, such as alkoxy, acyloxy, or aryloxy compounds. The selectively phosphorylated derivatives of cyclitols of the present invention also include the —OMe derivatives, such as pinitol, quebrachitol and bornesitol; cyclohexane-pentols in which one of the hydroxyl groups has been removed, such as quercitol; and cyclohexane-tetrols, wherein two hydroxyl groups have been removed. These compounds may also be prepared in the form of salts as indicated above.

The synthetic routes for preparation of selectively phosphorylated cyclitols disclosed by this invention are summarized in Schemes 1, 2, 3, and 4. Schemes 1, 2 and 3 show the preparation of polyphosphorylated cyclitols containing free hydroxyl, alkoxy, aryloxy and acyloxy groups. Scheme 4 shows the preparation of a protected 2,4,6-trisphosphate. In specific cases, the nature of the protecting groups or the order of the above reactions may have to be altered to reach desired products. These changes to the general synthetic schemes will be well understood by one of skill in the art. These synthetic routes are applicable to all conformational isomers of inositol.

In Schemes 1 and 2 a protected diol cyclitol derivative is reacted with NaH, DMF and an alkyl iodide or aryl bromide to obtain a dialkyl or diaryl ether. The dialkyl or diaryl ether is then reacted with trifluoroacetic acid to yield a a tetrol. Next, the tetrol is converted to tetrakisphosphate by reacting the tetrol with tetrazole in acetonitrile and dibenzyl N,N-diisopropylphosphoramidite, followed by addition of m-choro-perbenzoic acid in CH₂Cl₂. The tetrakisphosphate is then hydrogenated using a palladium catalyst to prepare the corresponding sodium salt.

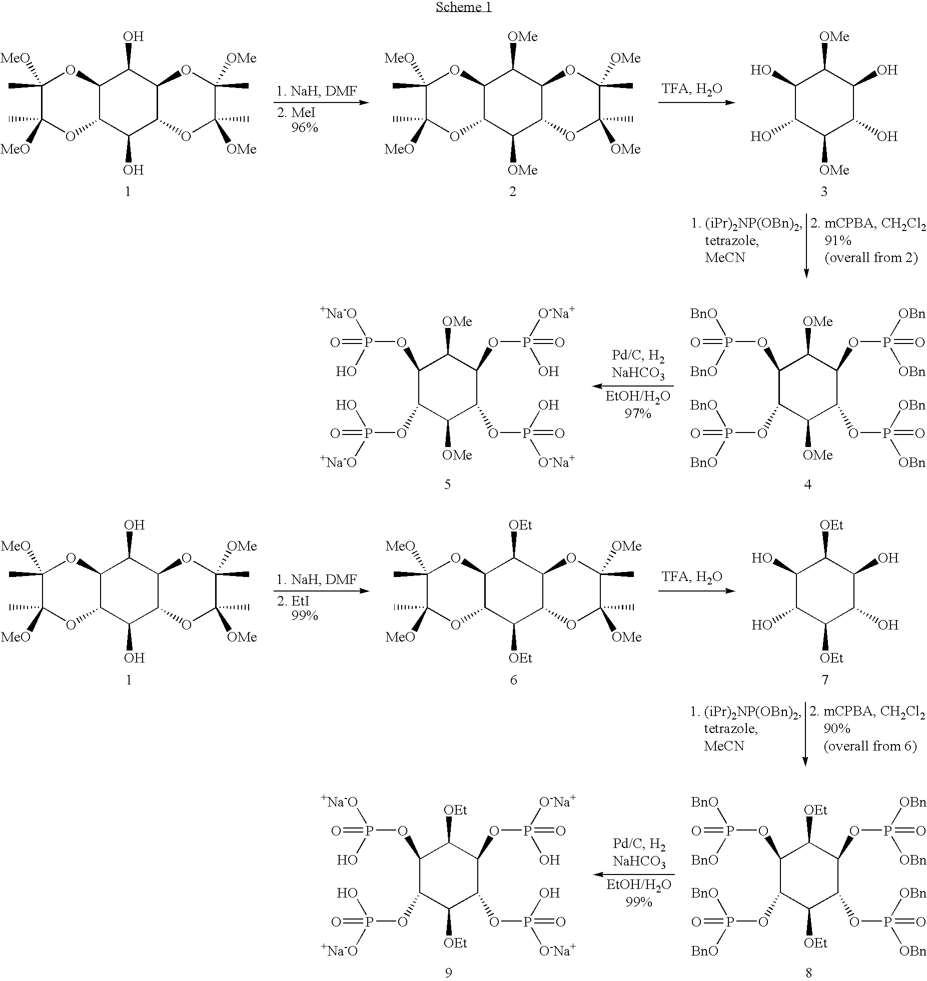

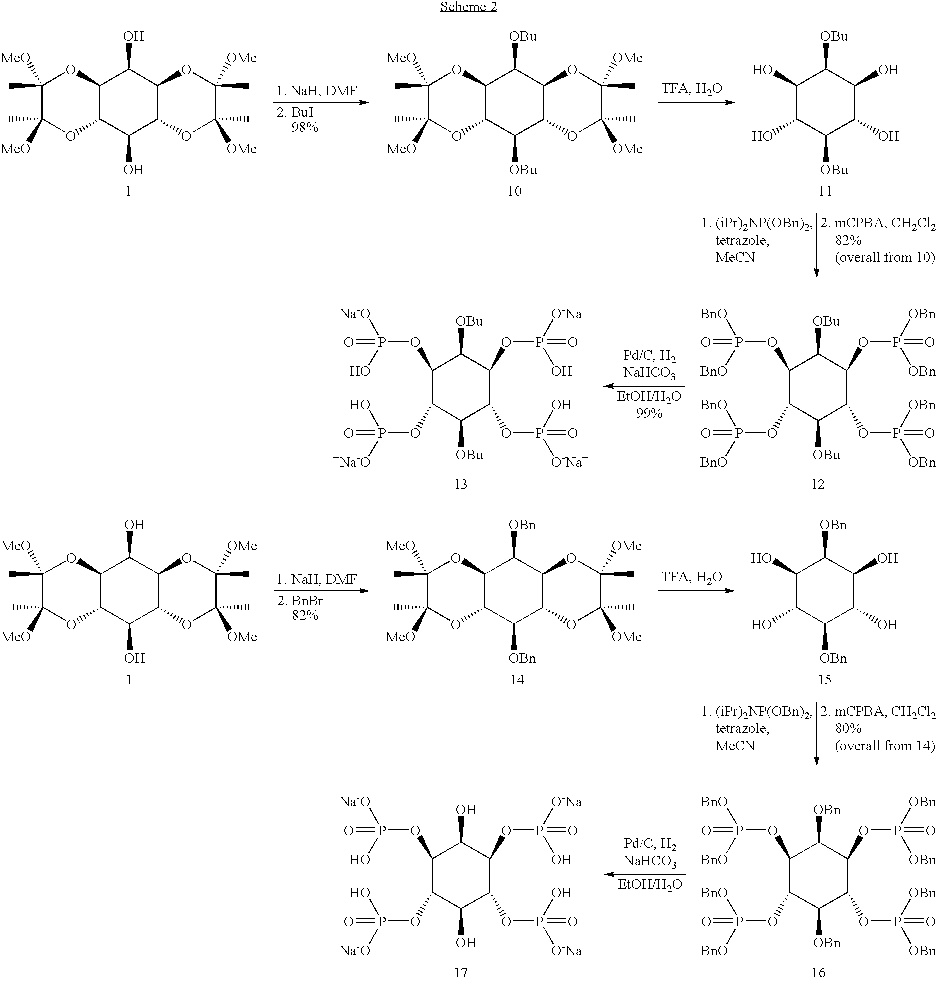

In Scheme 3, tetrazole is added to a 2,4,6-O-triacyl-inositol, followed by dibenzyl N,N diisopropylphosphoramidite and m-chloroperbenzoic acid to form the compound 10 (shown in Scheme 3). Next compound 10 is hydrogenated using a palladium catalyst to form a hexasodium 1,3,5-(2,4,6-tri-O-acyl)-inositol trisphosphate.

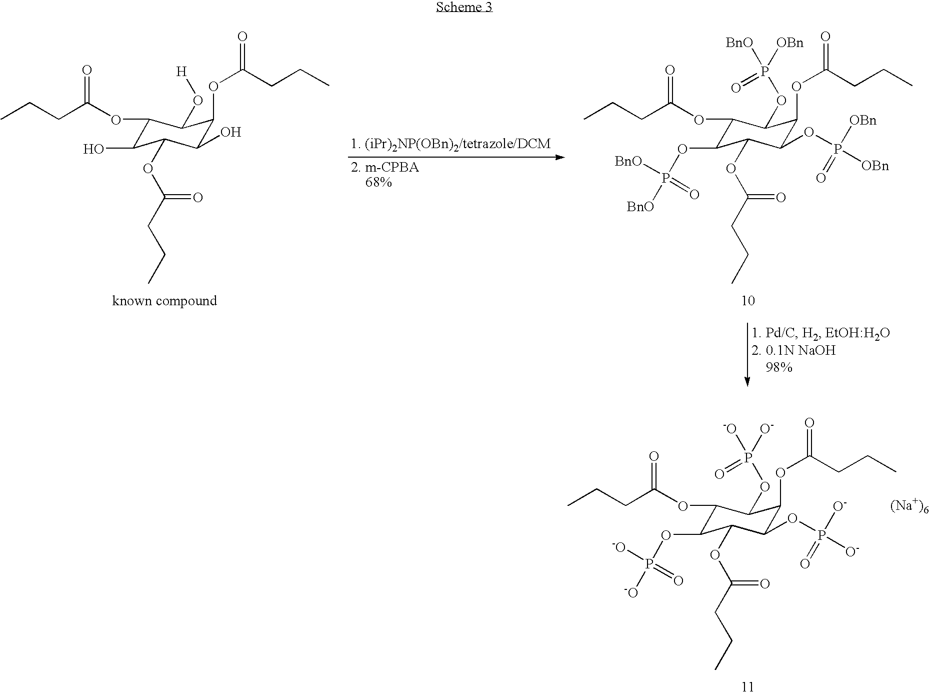

In Scheme 4, an inositol monoorthoformate is reacted with tetazole and dibenzyl N,N-diisopropylphosphoramidite and m-chloroperbenzoic acid to yield compound 8 (shown in Scheme 4). Compound 8 is hydrogenated using palladium catalyst to yield an orthoformate of 2,4,6-trisphosphate inositol.

It also is possible to derive polyphosphate cyclitol derivatives from hydrolysis and alchololysis of their corresponding pyrophosphate derivatives as described below under Scheme 6.

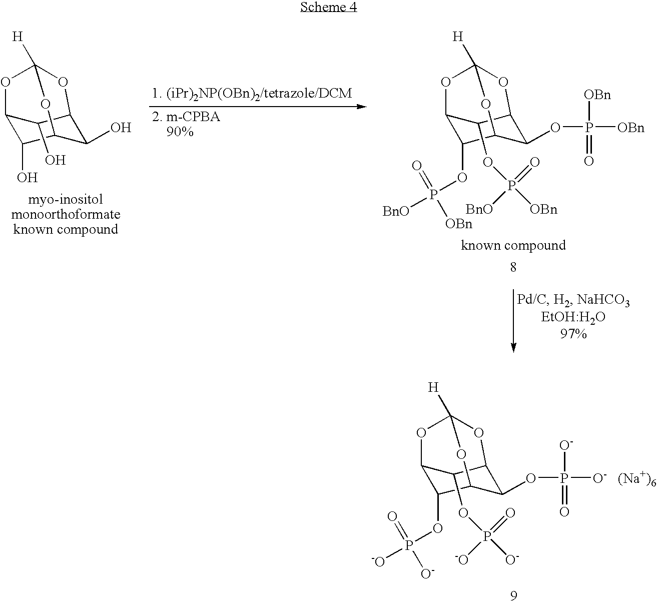

### Pyrophosphates of Cyclitols

The cyclitol polyphosphates described above can be converted into derivatives containing cyclic pyrophosphate groups by dehydration, using agents such as dicyclohexylcarbodiimide or related agents. This conversion may be total or yield compounds containing both phosphate and pyrophosphate functional groups. The compounds obtained are best isolated, purified and kept as their sodium salts. Other salts, such as ammonium salts, or salts of alkali earth metals, alkaline earth metals, or organic cations, may be prepared and serve a similar purpose. The fully phosphorylated inositol compounds may be used to derive compounds containing one, two or three pyrophosphate derivates, such as the trispyrophosphates of (+) or (−)-chiro-inositol, epi-inositol, scyllo-inositol, allo-inositol, muco-inositol, neo-inositol or myo-inositol.

The synthetic routes for preparation of pyrophosphate derivatives disclosed by this invention are summarized in Schemes 5, 6 and 7. Scheme 5 shows the preparation of a hexasodium trispyrophosphate of scyllo-inositol. Scheme 6 shows how hydrolysis and alcoholysis of tripyrophosphates of cylicotols can yield bispyrophosphates and polyphosphate derivatives in a non-stereoselective manner. Scheme 7 shows how a bispyrophosphate cyclitol can be prepared in a stereoselective manner. In specific cases, modifying the order of steps or reagents may be needed to reach the desired product. These changes to the general synthetic schemes will be well understood by one of skill in the art. These synthetic routes are applicable to all conformational isomers of inositol.

In Scheme 5 an inositol is reacted with tetrazole, dibenzyl N,N-diisopropylphosphoramidite to yield a inositol hexakis(dibenzyl phosphate). The inositol hexakis(dibenzyl phosphate) is then hydrogenated using a palladium catalyst to yield an inositiol hexakisphosphate. The debenzylated product is dissolved in triethylammonium to form a hexatriethylammonium salt of the inositol hexakisphosphate. The inositol hexakisphosphate salt is then reacted with 1,3-dicyclohexylcarbodiimide to yield the 1,2:3,4:5,6-trispyrophosphate hextriethylammonium salt of scyllo-inositol. This salt is then transformed into the corresponding hexasodium salt by exchange over a Dowex resin in its sodium form.

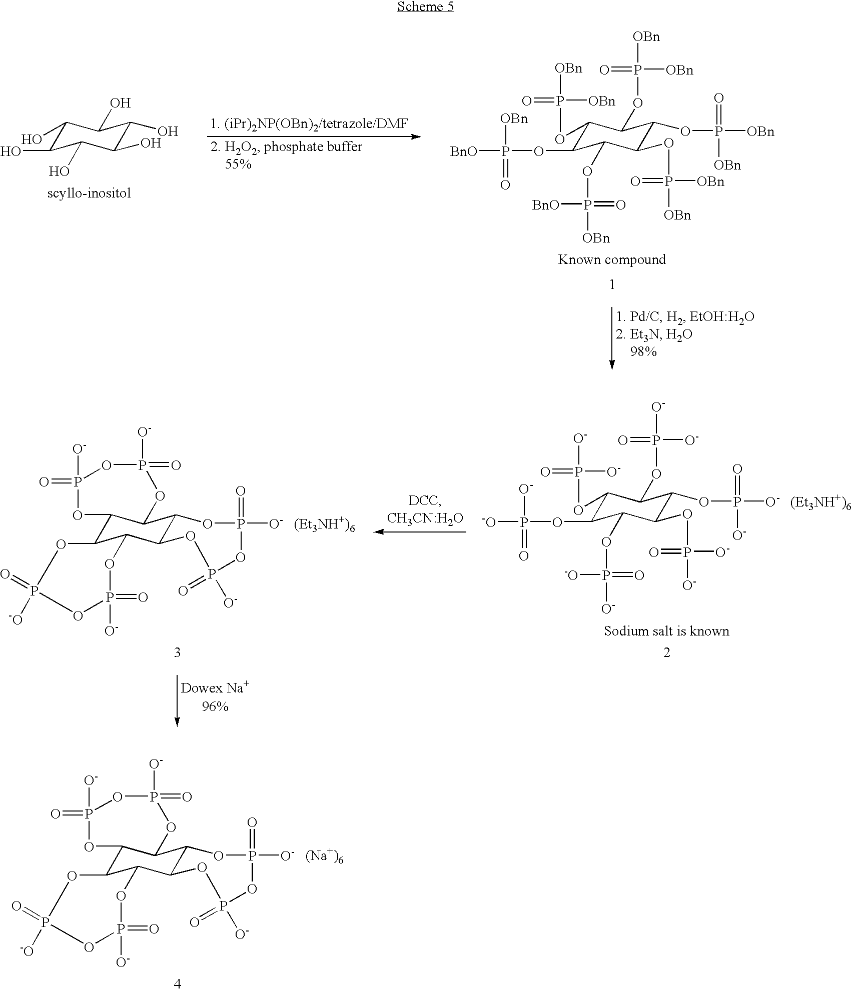

In Scheme 6, an inositol trispyrophosphate is passed over a Dowex 50WX8-200 column, and the acid fractions are pooled. After completion of the reaction the pH is adjusted to approximately 7 to yield a mixture of partially phosphorylated hydrolyzed product. Alternatively, the inositol trispyrophosphate can be reacted with acetyl chloride in the presence of an alcohol to yield a mixture of open pyrophosphate product as shown as depicted by compounds 6 and 7 of Scheme 6.

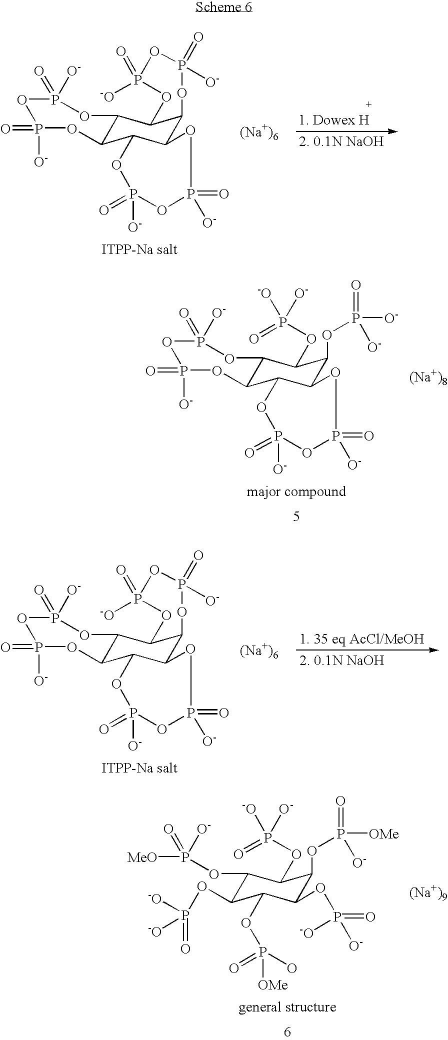

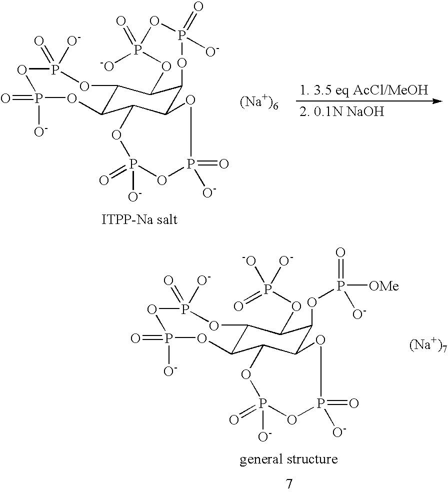

In Scheme 7, myo-inositol is condensed with cyclohexanone in the presence of PTSA to get a 1,2-cyclohexylidine myo-inositol which is treated with benzyl bromide and NaH to get a fully protected myo-inositol. Then the cyclohexylidine group is removed, followed by acylation to obtain a diacylated product. Next, debenzylation gives a tetrol, which is phosphorylated followed by oxidation resulting in a tetrakis(dibenzyl phosphate) derivative. Debenzylation with Pd/C in the presence of N,N-dimethylcyclohexyl amine followed by condensation with DCC results in a bispyrophosphate derivative. Saponification of bispyrophosphate derivative, followed by phosphorylation/oxidation results in a benzyl protected bispyrophosphate bisphosphate derivative. Finally, debenzylaion followed by sodium ion exchange results in the desired sodium salt of bispyrophosphate bisphosphate derivative 5 (Scheme 7). A similar synthetic strategy can also be used to prepare derivatives containing only one pyrophosphate group and four phosphate and/or phosphate ester groups.

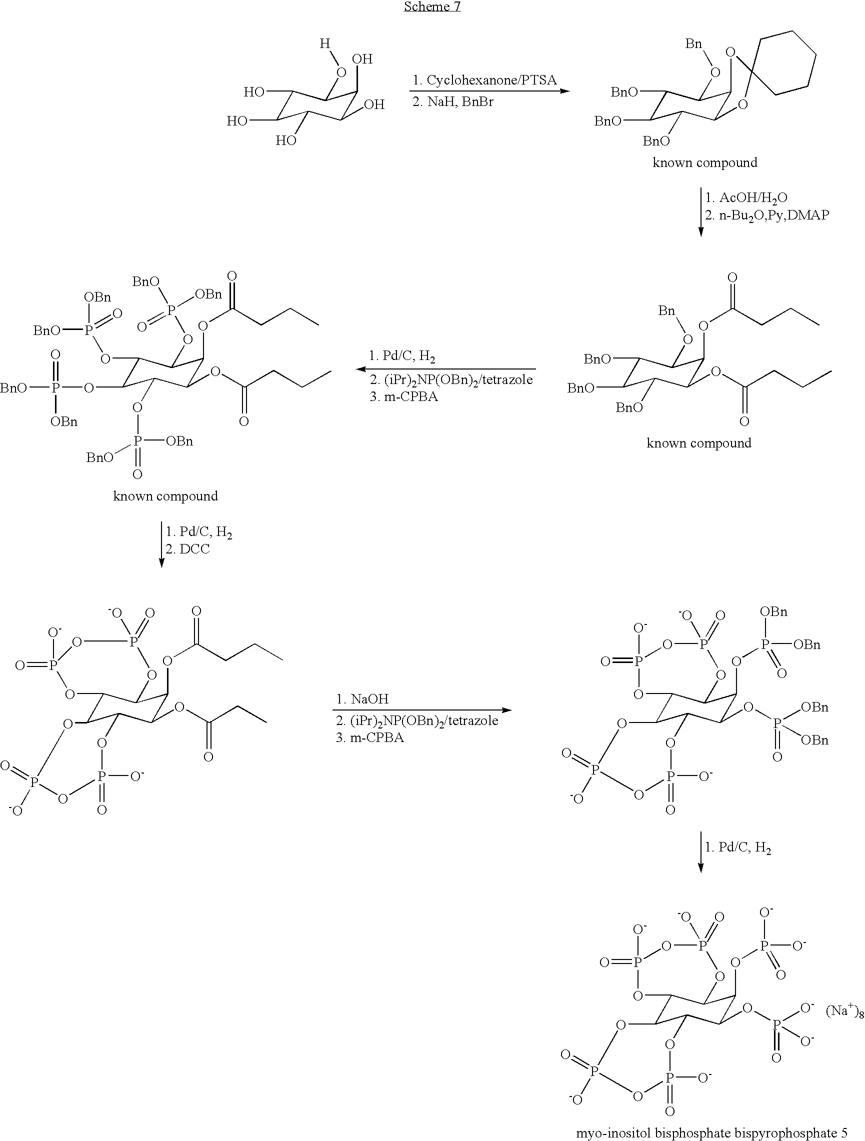

### Phosphorimide and Thiopyrophosphate Derivatives of Cyclitols

The cyclitol pyrophosphates, in particular the inositol trispyrophosphates, may be converted to their corresponding phosphorimides or thiopyrophosphates by a sequence of opening/closing reactions. For example, the cyclic pyrophosphate(s) may be opened with an amine of the general formula R—NH₂ to obtain a phosphoramidate, followed by closing the phosphoramidate with an agent like DCC to yield the corresponding phosphorimide. Alternatively, the cyclic pyrophosphate(s) with a metal sulfide (such as NaSH or Na₂S) to form a compound containing a mix of thiophosphate (—PO₂—SH) and phosphate groups (PO₃), and then closed back to the cyclic form, —PO₂—S—PO₂—, using a dehydrating agent to yield the thiopyrophosphate. The general structure of these compounds is provided in Formula I:

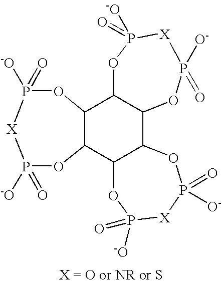

wherein X═O designates a trispyrophosphate, X═NR designates a trisphosphorimide, and X═S designates a tristhiopyrophosphate. For the phosphorimides, the R can be an H, and organic residue, a hydrocarbon chain of the form CₙH₂ₙ₊₁, or a chain or group containing heteroatoms, such as oxygen.

Where necessary in any of the synthetic procedures described herein, appropriate protecting groups may be used. Examples of protection groups can be found in the literature including “Protective Groups in Organic Synthesis—Third Edition (T. W. Greene, P. G. M Wuts, Wiley-Interscience, New York, N.Y., 1999). The present invention will be understood more readily by reference to the following examples, which are provided by way of illustration and are not intended to be limiting of the present invention.

### EXPERIMENTAL EXAMPLES

### 1,6:3,4-Bis-[O-(2,3-dimethoxybutane-2,3-diyl)]-2,5-di-O-methyl-myo-inositol (Scheme 1, Compound 2)

Diol (Scheme 1, Compound 1) (490 mg, 1.2 mmol) was dried under high vacuum for 8 h. Then, dry DMF (10 mL) was added under a N₂ atmosphere and the resulting suspension was cooled to 0° C. 90% NaH (120 mg, 4.8 mmoles) was added in one portion and the obtained slurry was stirred at the same temperature for 1 h. Methyl iodide (260 μL, 4.2 mmol) was added dropwise and the mixture was left to stir at room temperature for 12 h. Then, MeOH (300 μL) was slowly added and the mixture was left to stir at room temperature for 1 h. CH₂Cl₂ (25 mL) was added and the reaction mixture was washed with water (25 mL). The aqueous phase was back extracted with CH₂Cl₂ (25 mL) and the compined organic phases were washed with saturated brine (25 mL) and dried (MgSO₄). The solvents were removed under reduced pressure (30-55° C.) and the residue was purified by flash column chromatography (heptanes 30% ethyl acetate in heptanes) to yield dimethyl ether (Scheme 1, Compound 2) (500 mg, 96%) as a white solid. ¹H NMR (CDCl₃, 400 MHz): δ 3.99 (dd, J=10.2, 9.6 Hz, 2H), 3.63 (s, 3H), 3.57 (s, 3H), 3.55 (t, J=2.3 Hz, 1H), 3.52 (dd, J=10.2 Hz, 2.3 Hz, 2H), 3.28 (t, J=9.6 Hz, 1H, obscured), 3.28 (s, 6H), 3.25 (s, 6H), 1.30 (s, 6H), 1.29 (s, 6H); ¹³C NMR (CDCl₃, 100 MHz): δ 99.5, 98.9, 79.9, 77.8, 69.7, 69.4, 60.8, 60.4, 47.9, 47.7, 17.8, 17.6; HRMS (ESI): m/e Calcd for C₂₀H₃₆NaO₁₀ [(M+Na)⁺]: 459.2201, Found: 459.2203.

### 2,5-Di-O-methyl-myo-inositol (Scheme 1, Compound 3)

Dimethyl ether (Scheme 1, Compound 2) (470 mg, 1.1 mmol) was dissolved in aqueous 90% trifluoroacetic acid (5 mL) and the mixture was stirred at room temperature for 2 h. After the volatiles were removed under reduced pressure (40° C.) absolute ethanol (10 mL) was added and the solvent was again removed under reduced pressure. This sequence was repeated three times and yielded a tetrol (Scheme 1, Compound 3) (220 mg) as a white solid. This material was used in the next reaction without any further purification. mp 268-270 (EtOH); ¹H NMR (D₂O, 400 MHz): δ 3.64 (t, J=2.6 Hz, 1H), 3.57-3.47 (m, 4H), 3.50 (s, 3H), 3.49 (s, 3H), 2.93 (t, J=8.9 Hz, 1H); ¹³C NMR (D₂O, 100 MHz): δ 84.1, 82.5, 71.8, 71.5, 62.1, 59.5; HRMS (ESI): m/e Calcd for C₈H₁₆LiO₆ [(M+Li)⁺]: 215.1102, Found: 215.1133.

### Octabenzyl 1,3,4,6-(2,5-di-O-methyl-myo-inosityl)tetrakisphosphate (Scheme 1, Compound 4)

Tetrol (Scheme 1, Compound 3) (220 mg) was dried under high vacuum for 24 h. Then, a 0.45 M solution of tetrazole in acetonitrile (28.3 mL, 12.7 mmol) and dibenzyl N,N-diisopropylphosphoramidite (2.3 mL, 6.8 mmol) were added under a N₂ atmosphere at room temperature. The resulting slurry was vigorously stirred at room temperature for 24 h. CH₂Cl₂ (10 mL) was added and the mixture was cooled to −40° C. A solution of 70% m-chloro-perbenzoic acid (2.25 g, 9.1 mmol) in CH₂Cl₂ (14 mL) was added dropwise and the mixture was left to stir at 0° C for 5 h. Then, the mixture was diluted with CH₂Cl₂ (120 mL) and successively washed with a 10% aqueous solution of sodium sulphite (2×80 mL), saturated aqueous solution of sodium bicarbonate (2×60 mL), H₂O (60 mL) and saturated brine (60 mL). The organic phase was dried (MgSO₄) and the solvents were removed under reduced pressure (30° C.). The obtained residue was purified by flash column chromatography (heptanes 60% ethyl acetate in heptanes) to yield tetrakisphosphate (Scheme 1, Compound 4) (1.20 g, 91% overall from 2) as a thick colorless oil. ¹H NMR (CDCl₃, 400 MHz): δ 7.35-7.26 (m, 40H), 5.16-5.00 (m, 16H), 4.94 (q, J=9.4 Hz, 2H), 4.50 (bs, 1H), 4.25 (ddd, ³J_{HH}=9.6, 2.3 Hz, ³J_{HP}=7.6 Hz, 2H), 3.57 (s, 3H), 3.50 (s, 3H), 3.25 (t, J=9.3 Hz, 1H); ¹³C NMR (CDCl₃, 100 MHz): δ 135.4 (d, ³J_{CP}=6.9 Hz, 2C), 135.1 (d, ³J_{CP}=6.9 Hz), 135.0 (d, ³J_{CP}=6.9 Hz), 128.00, 127.98, 127.9, 127.75, 127.71, 127.6, 127.5, 127.32, 127.25, 80.2, 77.6, 76.2 (t, ²J_{CP}=6.9 Hz), 75.2, 69.3 (d, ³J_{CP}=5.3 Hz), 69.0 (d, ³J_{CP}=5.3 Hz), 68.8 (d, ³J_{CP}=6.1 Hz), 68.7 (d, ³J_{CP}=5.3 Hz), 59.6, 59.2; ³¹P NMR (162 MHz): δ −1.2, −1.7; HRMS (ESI): m/e Calcd for C₆₄H₆₈NaO₁₈P₄ [(M+Na)⁺]: 1271.3248, Found: 1271.3434.

### Tetrasodium 1,3,4,6-(2,5-di-O-methyl-myo-inosityl)tetrakisphosphate (Scheme 1, Compound 5)

Tetrakisphosphate (Scheme 1, Compound 4) (130 mg, 0.1 mmol) was dissolved in an 1:1 mixture of ethanol and H₂O (10 mL). Sodium bicarbonate (34 mg, 0.4 mmol) was added to the resulting emulsion followed by 10% Pd/C (100 mg). This mixture was left to vigorously stir under a H₂ atmosphere (1 Atm) at room temperature for 24 h. The catalyst was removed by filtration through an LCR/PTFE hydrophillic membrane (0.5 μm), the latter was washed with an 1:1 mixture of ethanol and H₂O (3×10 mL). The combined filtrates were evaporated under reduced pressure (60° C.) and the obtained residue was dried under high vacuum for 24 h to give tetrasodium salt (Scheme 1, Compound 5) as a glassy white solid (60 mg, 97%). ¹H NMR (D₂O, 400 MHz): δ 4.27 (q, J=9.1 Hz, 2H), 4.05 (t, J=10.1 Hz, 2H), 3.88 (s, 1H), 3.60 (s, 3H), 3.54 (s, 3H), 3.26 (t, J=9.3 Hz, 1H); ¹³C NMR (D₂O, 100 MHz): δ 83.2, 81.2, 75.6, 74.0, 61.9, 59.9.

### 1,6:3,4-Bis-[O-(2,3-dimethoxybutane-2,3-diyl)]-2,5-di-O-ethyl-myo-inositol (Scheme 1, Compound 6)

Diol (Scheme 1, Compound 1) (490 mg, 1.2 mmol) was dried under high vacuum for 8 h. Then, dry DMF (10 mL) was added under a N₂ atmosphere and the resulting suspension was cooled to 0° C. 90% NaH (120 mg, 4.8 mmoles) was added in one portion and the obtained slurry was stirred at the same temperature for 1 h. Ethyl iodide (340 μL, 4.2 mmol) was added dropwise and the mixture was left to stir at room temperature for 12 h. Then, MeOH (300 μL) was slowly added and the mixture was left to stir at room temperature for 1 h. CH₂Cl₂ (25 mL) was added and the reaction mixture was washed with water (25 mL). The aqueous phase was back extracted with CH₂Cl₂ (25 mL) and the compined organic phases were washed with saturated brine (25 mL) and dried (MgSO₄). The solvents were removed under reduced pressure (30-55° C.) and the residue was purified by flash column chromatography (heptanes 30% ethyl acetate in heptanes) to yield diethyl ether (Scheme 1, Compound 6) (550 mg, 99%) as a thick pale yellow oil. ¹H NMR (CDCl₃, 400 MHz): δ 3.92 (t, J=10.0 Hz, 2H), 3.74 (q, J=7.1 Hz, 2H), 3.69 (q, J=7.0 Hz, 2H), 3.57 (t, J=2.2 Hz, 1H), 3.40 (dd, J=10.2, 2.2 Hz, 2H), 3.22 (t, J=9.8 Hz, 1H), 3.20 (s, 6H), 3.16 (s, 6H), 1.20 (2×s, 2×6H), 1.10 (t, J=7.0 Hz, 6H); ¹³C NMR (CDCl₃, 100 MHz): δ 99.3, 98.7, 78.5, 76.1, 69.7, 69.2, 68.2, 67.3, 47.6, 47.5, 17.7, 17.5, 15.7, 15.5; HRMS (ESI): m/e Calcd for C₂₂H₄₀NaO₁₀ [(M+Na)⁺]: 487.2514, Found: 487.2466.

### 2,5-Di-O-ethyl-myo-inositol (Scheme 1, Compound 7)

Diethyl ether (Scheme 1, Compound 6) (540 mg, 1.2 mmol) was dissolved in aqueous 90% trifluoroacetic acid (5 mL) and the mixture was stirred at room temperature for 2 h. After the volatiles were removed under reduced pressure (40° C.) absolute ethanol (10 mL) was added and the solvent was again removed under reduced pressure. This sequence was repeated three times and yielded a tetrol (Scheme 1, Compound 7) (270 mg) as a white solid. This material was used in the next reaction without any further purification. ¹H NMR (D₂O, 400 MHz): δ 3.77-3.63 (m, 5H), 3.52 (t, J=9.6 Hz, 2H), 3.41 (dd, J=10.2, 2.6 Hz, 2H), 2.96 (t, J=9.2 Hz, 1H); 1.08 (t, J=7.0 Hz, 3H), 1.05 (t, J=7.0 Hz, 3H); ¹³C NMR (D₂O, 100 MHz): δ 82.9, 80.2, 72.1, 71.4, 69.9, 68.5, 14.7; HRMS (ESI): m/e Calcd for C₁₀H₂₀NaO₆ [(M+Na)⁺]: 259.1152, Found: 259.1148.

### Octabenzyl 1,3,4,6-(2,5-di-O-ethyl-myo-inosityl)tetrakisphosphate (Scheme 1, Compound 8)

Tetrol (Scheme 1, Compound 7) (270 mg) was dried under high vacuum for 24 h. Then, 0.45 M solution of tetrazole in acetonitrile (30.5 mL, 13.7 mmol) and dibenzyl N,N-diisopropylphosphoramidite (2.5 mL, 7.3 mmol) were added under a N₂ atmosphere at room temperature. The resulting slurry was vigorously stirred at room temperature for 24 h. CH₂Cl₂ (10 mL) was added and the mixture was cooled to −40° C. A solution of 70% m-chloro-perbenzoic acid (2.42 g, 9.8 mmol) in CH₂Cl₂ (15 mL) was added dropwise and the mixture was left to stir at 0° C. for 5 h. Then, the mixture was diluted with CH₂Cl₂ (150 mL) and successively washed with a 10% aqueous solution of sodium sulphite (2×100 mL), saturated aqueous solution of sodium bicarbonate (2×75 mL), H₂O (75 mL) and saturated brine (75 mL). The organic phase was dried (MgSO₄) and the solvents were removed under reduced pressure (30° C.). The obtained residue was purified by flash column chromatography (heptanes 60% ethyl acetate in heptanes) to yield tetrakisphosphate (Scheme 1, Compound 8) (1.31 g, 90% overall from 6) as a thick pale yellow oil. ¹H NMR (CDCl₃, 400 MHz): δ 7.30-7.21 (m, 40H), 5.08-4.99 (m, 16H), 4.90 (q, J=9.4 Hz, 2H), 4.55 (bs, 1H), 4.15 (ddd, ³J_{HH}=9.6, 2.0 Hz, ³J_{HP}=7.6 Hz, 2H), 3.75 (q, J=7.0 Hz, 2H), 3.71 (q, J=7.0 Hz, 2H), 3.27 (t, J=9.4 Hz, 1H), 1.07 (t, J=7.0 Hz, 3H), 1.06 (t, J=7.0 Hz, 3H); ¹³C NMR (CDCl₃, 100 MHz): δ 135.9 (d, ³J_{CP}=7.6 Hz), 135.8 (d, ³J_{CP}=6.9 Hz), 135.5 (d, ³J_{CP}=6.9 Hz), 135.4 (d, ³J_{CP}=6.9 Hz), 128.34, 128.32, 128.30, 128.23, 128.21, 128.12, 128.07, 128.01, 128.0, 127.82, 127.79, 127.77, 127.73, 127.67, 78.9, 77.6 (t, ²J_{CP}=7.6 Hz), 76.5, 75.6 (t, ²J_{CP}=4.2 Hz), 69.6 (d, ³J_{CP}=6.1 Hz), 69.5, 69.4 (d, ³J_{CP}=5.3 Hz), 69.3 (d, ³J_{CP}=5.3 Hz), 69.2 (d, ³J_{CP}=5.3 Hz), 67.7, 15.5, 14.7; ³¹P NMR (162 MHz): δ −1.5, −1.7; HRMS (ESI): m/e Calcd for C₆₆H₇₃O₁₈P₄ [(M+H)⁺]: 1277.3742, Found: 1277.3854.

### Tetrasodium 1,3,4,6-(2,5-di-O-ethyl-myo-inosityl)tetrakisphosphate (Scheme 1, Compound 9)

Tetrakisphosphate (Scheme 1, Compound 8) (320 mg, 0.25 mmol) was dissolved in an 1:1 mixture of ethanol and H₂O (20 mL). Sodium bicarbonate (84 mg, 1 mmol) was added to the resulting emulsion followed by 10% Pd/C (250 mg). This mixture was left to vigorously stir under a H₂ atmosphere (1 Atm) at room temperature for 22 h. The catalyst was removed by filtration through an LCR/PTFE hydrophillic membrane (0.5 μm), the latter was washed with an 1:1 mixture of ethanol and H₂O (3×10 mL). The combined filtrates were evaporated under reduced pressure (60° C.) and the obtained residue was dried under high vacuum for 24 h to give tetrasodium salt (Scheme 1, Compound 9) as a glassy white solid (160 mg, 99%).¹H NMR (D₂O, 400 MHz): δ 4.26 (q, J=7.0 Hz, 2H), 4.04 (t, J=8.8 Hz, 2H), 4.00 (s, 1H), 3.78 (q, J=7.0 Hz, 2H), 3.74 (q, J=7.0 Hz, 2H), 3.31 (t, J=9.4 Hz, 1H), 1.11 (t, J=7.0 Hz, 3H), 1.10 (t, J=7.0 Hz, 3H); ¹³C NMR (D₂O, 100 MHz): δ 80.4, 78.5, 76.5, 74.5, 69.9, 68.5, 14.8.

### 1,6:3,4-Bis-[O-(2,3-dimethoxybutane-2,3-diyl)]-2,5-di-O-butyl-myo-inositol (Scheme 2, Compound 10)

Diol (Scheme 2, Compound 1) (490 mg, 1.2 mmol) was dried under high vacuum for 8 h. Then, dry DMF (10 mL) was added under a N₂ atmosphere and the resulting suspension was cooled to 0° C. 90% NaH (120 mg, 4.8 mmoles) was added in one portion and the obtained slurry was stirred at the same temperature for 1 h. Butyl iodide (480 μL, 4.2 mmol) was added dropwise and the mixture was left to stir at room temperature for 12 h. Then, MeOH (300 μL) was slowly added and the mixture was left to stir at room temperature for 1 h. CH₂Cl₂ (25 mL) was added and the reaction mixture was washed with water (25 mL). The aqueous phase was back extracted with CH₂Cl₂ (25 mL) and the compined organic phases were washed with saturated brine (25 mL) and dried (MgSO₄). The solvents were removed under reduced pressure (30-55° C.) and the residue was purified by flash column chromatography (heptanes 30% ethyl acetate in heptanes) to yield dibutyl ether (Scheme 2, Compound 10) (610 mg, 98%) as a thick yellow oil. ¹H NMR (CDCl₃, 400 MHz): δ 3.95 (t, J=9.8 Hz, 2H), 3.73 (t, J=6.3 Hz, 2H), 3.66 (t, J=6.4 Hz, 2H), 3.56 (t, J=2.3 Hz, 1H), 3.42 (dd, J=10.2, 2.3 Hz, 2H), 3.24 (t, J=9.2 Hz, 1H), 3.23 (s, 6H), 3.20 (s, 6H), 1.55-1.46 (m, 4H), 1.42-1.32 (m, 4H), 1.24 (s, 12H), 0.87 (t, J=7.3 Hz, 3H), 0.87 (t, J=7.2 Hz, 3H); ¹³C NMR (CDCl₃, 100 MHz): δ 99.4, 98.8, 78.5, 76.5, 72.5, 72.0, 69.8, 69.3, 47.7, 47.5, 32.3, 32.2, 19.1, 19.0, 17.8, 17.5, 13.9, 13.7; HRMS (ESI): m/e Calcd for C₂₆H₄₈NaO₁₀ [(M+Na)⁺]: 543.3140, Found: 543.3112.

### 2,5-Di-O-butyl-myo-inositol (Scheme 2, Compound 11)

Dibutyl ether (Scheme 1, Compound 10) (600 mg, 1.2 mmol) was dissolved in aqueous 90% trifluoroacetic acid (5 mL) and the mixture was stirred at room temperature for 2 h. After the volatiles were removed under reduced pressure (40° C.) absolute ethanol (10 mL) was added and the solvent was again removed under reduced pressure. This sequence was repeated three times and yielded a tetrol (Scheme 2, Compound 11) (332 mg) as a white solid. This material was used in the next reaction without any further purification. HRMS (ESI): m/e Calculated for C₁₄H₂₈LiO₆ [(M+Li)⁺]: 299.2041, Found: 299.2056.

### Octabenzyl 1,3,4,6-(2,5-di-O-butyl-myo-inosityl)tetrakisphosphate (Scheme 2, Compound 12)

Tetrol (Scheme 2, Compound 11) (332 mg) was dried under high vacuum for 24 h. Then, 0.45 M solution of tetrazole in acetonitrile (30.1 mL, 13.6 mmol) and dibenzyl N,N-diisopropylphosphoramidite (2.4 mL, 7.2 mmol) were added under a N₂ atmosphere at room temperature. The resulting slurry was vigorously stirred at room temperature for 24 h. CH₂Cl₂ (10 mL) was added and the mixture was cooled to −40° C. A solution of 70% m-chloro-perbenzoic acid (2.4 g, 9.7 mmol) in CH₂Cl₂ (15 mL) was added dropwise and the mixture was left to stir at 0° C. for 5 h. Then, the mixture was diluted with CH₂Cl₂ (150 mL) and successively washed with a 10% aqueous solution of sodium sulphite (2×100 mL), saturated aqueous solution of sodium bicarbonate (2×75 mL), H₂O (75 mL) and saturated brine (75 mL). The organic phase was dried (MgSO₄) and the solvents were removed under reduced pressure (30° C.). The obtained residue was purified by flash column chromatography (heptanes 60% ethyl acetate in heptanes) to yield tetrakisphosphate (Scheme 2, Compound 12) (1.23 g, 82% overall from 10) as a thick pale yellow oil. ¹H NMR (CDCl₃, 400 MHz): δ 7.32-7.21 (m, 40 H), 5.11-4.98 (m, 16H), 4.90 (q, J=9.3 Hz, 2H), 4.54 (bs, 1H), 4.18 (ddd, ³J_{HH}=9.6, 2.0 Hz, ³J_{HP}=7.3 Hz, 2H), 3.67 (t, J=7.3 Hz, 2H), 3.66 (t, J=7.5 Hz, 2H), 3.28 (t, J=9.4 Hz, 1H), 1.52-1.40 (m, 4H), 1.27-1.21 (m, 2H), 1.08-1.03 (m, 2H), 0.80 (t, J=7.5 Hz, 3H), 0.69 (t, J=7.3 Hz, 3H); ¹³C NMR (CDCl₃, 100 MHz): δ 135.9 (d, ³J_{CP}=7.6 Hz), 135.8 (d, ³J_{CP}=6.9 Hz), 135.54 (d, ³J_{CP}=7.6 Hz), 135.48 (d, ³J_{CP}=7.6 Hz), 128.40, 128.38, 128.36, 128.33, 128.27, 128.26, 128.14, 128.09, 128.99, 127.94, 127.88, 127.77. 127.73, 127.71, 127.70, 127.68, 127.65, 79.2, 77.2 (t, ²J_{CP}=6.9 Hz), 76.6, 75.7 (t, ²J_{CP}=4.0 Hz), 73.9, 72.6, 69.6 (³J_{CP}=5.3 Hz), 69.4 (³J_{CP}=5.3 Hz), 69.3 (³J_{CP}=5.3 Hz), 69.2 (³J_{CP}=5.3 Hz), 32.1, 31.5, 18.9, 18.7, 13.9, 13.7; ³¹P NMR (162 MHz): δ −1.4, −1.7; HRMS (ESI): m/e Calculated for C₇₀H₈₀NaO₁₈P₄ [(M+Na)⁺]: 1355.4187, Found: 1355.4220.

### Tetrasodium 1,3,4,6-(2,5-di-O-butyl-myo-inosityl)tetrakisphosphate (Scheme 2, Compound 13)

Tetrakisphosphate (Scheme 2, Compound 12) (320 mg, 0.24 mmol) was dissolved in an 1:1 mixture of ethanol and H₂O (10 mL). Sodium bicarbonate (81 mg, 0.96 mmol) was added to the resulting emulsion followed by 10% Pd/C (240 mg). This mixture was left to vigorously stir under a H₂ atmosphere (1 Atm) at room temperature for 21 h. The catalyst was removed by filtration through an LCR/PTFE hydrophillic membrane (0.5 μm), the latter was washed with an 1:1 mixture of ethanol and H₂O (3×10 mL). The combined filtrates were evaporated under reduced pressure (60° C.) and the obtained residue was dried under high vacuum for 24 h to give tetrasodium salt (Scheme 2, Compound 13) as a glassy white solid (163 mg, 97%).¹H NMR (D₂O, 400 MHz): δ 4.36 (q, J=9.4 Hz, 2H), 4.08 (dt, J=9.9, 2.0 Hz, 2H), 4.06 (s, 1H), 3.81 (t, J=6.9 Hz, 2H), 3.75 (t, J=7.5 Hz, 2H), 3.39 (t, J=9.3 Hz, 1H), 1.61-1.52 (m, 4H), 1.39-1.23 (m , 4H), 0.87 (t, J=7.5 Hz, 3H), 0.85 (t, J=7.3 Hz, 3H); ¹³C NMR (D₂O, 100 MHz): δ 80.8, 78.9, 76.5, 74.7, 74.2, 72.8, 31.4, 18.65, 18.55, 13.5, 13.4.

### 2,5-Di-O-benzyl-1,6:3,4-bis-[O-(2,3-dimethoxybutane-2,3-diyl)]-myo-inositol (Scheme 2, Compound 14)

Diol (Scheme 2, Compound 1) (1.47 g, 3.6 mmol) was dried under high vacuum for 8 h. Then, dry DMF (30 mL) was added under a N₂ atmosphere and the resulting suspension was cooled to 0° C. 90% NaH (345 mg, 14.4 mmoles) was added in one portion and the obtained slurry was stirred at the same temperature for 1 h. Benzyl bromide (1.5 mL, 12.6 mmol) was added dropwise and the mixture was left to stir at 40° C. for 20 h. Then it was cooled to room temperature, MeOH (1 mL) was slowly added and the mixture was left to stir at room temperature for 1 h. CH₂Cl₂ (75 mL) was added and the reaction mixture was washed with water (75 mL). The aqueous phase was back extracted with CH₂Cl₂ (75 mL) and the compined organic phases were washed with saturated brine (75 mL) and dried (MgSO₄). The solvents were removed under reduced pressure (30-55° C.) and the residue was purified by flash column chromatography (heptanes 10% ethyl acetate in heptanes) to yield dibenzyl ether (Scheme 2, Compound 14) (1.74 g, 82%) as a white solid. ¹H NMR (CDCl₃, 400 MHz): δ 7.50 (d, J=7.9 Hz, 2H), 7.41 (d, J=7.9 Hz, 2H), 7.31-7.27 (m, 4H), 7.24-7.20 (m, 2H), 4.87 (s, 2H), 4.85 (s, 2H), 4.20 (t, J=9.8 Hz, 2H), 3.80 (bs, 1H), 3.58 (bd, J=10.5 Hz, 2H), 3.26 (s, 6H), 3.25 (t, J=9.4 Hz, 1H, obscured), 3.23 (s, 6H), 1.33 (s, 6H), 1.31 (s, 6H); ¹³C NMR (CDCl₃, 100 MHz): δ 139.5, 127.9, 127.7, 127.5, 127.4, 127.1, 126.8, 99.5, 98.9, 78.8, 76.0, 74.9, 73.7, 69.9, 69.2, 47.8, 47.7, 17.8, 17.6; HRMS (ESI): m/e Calcd for C₃₂H₄₄NaO₁₀ [(M+Na)⁺]: 611.2827, Found: 611.2824.

### 2,5-Di-O-benzyl-myo-inositol (Scheme 2, Compound 15)

Dibenzyl ether (Scheme 2, Compound 14) (2.07 g, 3.5 mmol) was dissolved in CH₂Cl₂ (2.8 mL). Aqueous 90% trifluoroacetic acid (14 mL) was added and the mixture was stirred at room temperature for 75 min. After the volatiles were removed under reduced pressure (40° C.) absolute ethanol (25 mL) was added and the solvent was again removed under reduced pressure. This sequence was repeated three times and yielded a tetrol (Scheme 2, Compound 15) (1.06 g) as a white solid. This material was used in the next reaction without any further purification. HRMS (ESI): m/e Calcd for C₂₀H₂₄NaO₆ [(M+Na)⁺]: 383.1456, Found: 383.1442.

### Octabenzyl 1,3,4,6-(2,5-di-O-benzyl-myo-inosityl)tetrakisphosphate (Scheme 2, Compound 16)

Tetrol (Scheme 2, Compound 15) (1.06 g) was dried under high vacuum for 24 h. Then, 0.45 M solution of tetrazole in acetonitrile (93 mL, 42 mmol) and dibenzyl N,N-diisopropylphosphoramidite (7.5 mL, 22.4 mmol) were added under a N₂ atmosphere at room temperature. The resulting slurry was vigorously stirred at room temperature for 24 h. CH₂Cl₂ (35 mL) was added and the mixture was cooled to −40° C. A solution of 70% m-chloro-perbenzoic acid (5.8 g, 23.4 mmol) in CH₂Cl₂ (50 mL) was added dropwise and the mixture was left to stir at 0° C. for 5 h. Then, the mixture was diluted with CH₂Cl₂ (500 mL) and successively washed with a 10% aqueous solution of sodium sulphite (2×350 mL), saturated aqueous solution of sodium bicarbonate (2×250 mL), H₂O (250 mL) and saturated brine (250 mL). The organic phase was dried (MgSO₄) and the solvents were removed under reduced pressure (30° C.). The obtained residue was purified by flash column chromatography (heptanes 50% ethyl acetate in heptanes) to yield tetrakisphosphate (Scheme 2, Compound 16) (3.90 g, 80% overall from Scheme 2, Compound 14)) as a thick pale yellow oil. ¹H NMR (CDCl₃, 400 MHz): δ 7.44 (bd, J=6.4 Hz, 2H), 7.28-7.11 (m, 44H), 7.00 (bd, J=7.8 Hz, 4H), 5.09 (q, J=9.4 Hz, 2H), 5.04-4.94 (m, 10H), 4.91-4.86 (m, 6H), 4.78 (bs, 3H), 4.70 (dd, J=11.7, 9.1 Hz, 2H), 4.29 (ddd, ³J_{HH}=9.7, 2.1 Hz, ³J_{HP}=7.4 Hz, 2H), 3.51 (t, J=9.4 Hz, 1H); ¹³C NMR (CDCl₃, 100 MHz): δ 138.0, 137.8, 135.8 (d, ³J_{CP}=7.6 Hz), 135.7 (d, ³J_{CP}=6.9 Hz), 135.5 (d, ³J_{CP}=6.9 Hz), 135.4 (d, ³J_{CP}=6.9 Hz), 128.43, 128.40, 128.3, 128.22, 128.18, 128.11, 128.08, 128.0, 127.95, 127.91, 127.7, 127.4, 127.3, 127.2, 127.1, 78.9, 77.2 (t, ²J_{CP}=6.9 Hz), 77.1, 75.8, 75.6 (d, ²J_{CP}=5.3 Hz), 73.8, 69.8 (d, ³J_{CP}=6.1 Hz), 69.5 (d, ³J_{CP}=5.3 Hz), 69.3 (d, ³J_{CP}=6.1 Hz), 69.2 (d, ³J_{CP}=5.3 Hz); HRMS (ESI): m/e Calcd for C₇₆H₇₆NaO₁₈P₄ [(M+Na)⁺]: 1423.3874, Found: 1423.3884.

### Tetrasodium 1,3,4,6-myo-inosityl tetrakisphosphate (Scheme 2, Compound 17)

The octabenzylated tetrakisphosphate (Scheme 2, Compound 16) (380 mg, 0.27 mmol) was hydrogenolyzed by dissolution in an 1:1 mixture of ethanol and H₂O (20 mL). Sodium bicarbonate (91 mg, 1.08 mmol) was added to the resulting emulsion followed by 10% Pd/C (270 mg). This mixture was left to vigorously stir under a H₂ atmosphere (1 Atm) at room temperature until the starting material was fully consumed.

### Hexabenzyl 1,3,5-(2,4,6-tri-O-butyryl-myo-inosityl)trisphosphate (Scheme 3, Compound 10)

To a solution of 2,4,6-tri-O-butyryl-myo-inositol³¹ (230 mg, 0.58 mmol, 1 eq) in DCM (5 mL), tetrazole in CH₃CN (0.45 M, 5.89 mL, 2.65 mmol, 4.5 eq) was added at room temperature followed by dibenzyl N,N-diisopropylphosphoramidite (0.87 mL, 2.65 mmol, 4.5 eq). After being stirred for 24 h, the reaction mixture was cooled to −40° C., m-chloroperbenzoic acid (508 mg, 2.94 mmol, 5 eq) was added portionwise and stirred from −40° C. to room temperature for 12 h. The reaction mixture was diluted with EtOAc, washed with 1N HCl, saturated NaHCO₃, brine, dried (Na₂SO₄) and concentrated in vacuo. The crude was purified by silica gel chromatography (EtOAc/n-heptane, 10:90 to 90:10) afford 471 mg (68%) of hexabenzyl 1,3,5-(2,4,6-tri-O-butyryl-myo-inosityl) trisphosphate (Scheme 3, Compound 10). TLC (SiO₂): R_{f}=0.24 (EtOAc/n-heptane, 60:40); ¹H NMR (CDCl₃, 400 MHz, 25° C.): δ=7.34-7.23 (m, 30 H), 5.94 (t, J=2.8 Hz, 1 H), 5.58 (t, J=9.9 Hz, 2 H), 5.03-4.89 (m, 12 H), 4.43 (dt, J=10.0, 2.8 Hz, 2 H), 4.39 (q, J=9.5 Hz, 1 H), 2.38 (t, J=7.4 Hz, 2 H), 2.06 (t, J=7.4 Hz, 2 H), 2.05 (t, J=7.4 Hz, 2 H), 1.67-1.58 (m, 2 H), 1.39-1.29 (m, 4 H), 0.93 (t, J=7.4 Hz, 3 H), 0.65 (t, J=7.5 Hz, 6 H); ¹³C NMR (CDCl₃, 100 MHz, 25° C.): δ=172.7, 172.0, 135.5 (d, ³J_{CP}=7.2 Hz), 135.4 (d, ³J_{CP}=6.0 Hz), 135.3 (d, ³J_{CP}=5.9 Hz), 128.63, 128.59, 128.0, 127.96, 127.95, 75.9 (d, J=5.6 Hz), 72.9 (d, J=5.1 Hz), 69.8 (d, J=5.7 Hz), 69.63 (d, J=6.2 Hz), 69.56 (d, J=6.1 Hz), 69.4 (bs), 35.9, 35.5, 18.5, 17.5, 13.5; ³¹P NMR (CDCl₃, 162 MHz, 25° C.): δ=−1.50, −1.73; HRMS (ESI-MS): m/z: calcd for C₆₀H₆₉O₁₈P₃Na₂: 608.1741 [M+2Na]⁺²; found: 608.1704.

### Hexasodium 1,3,5-(2,4,6-tri-O-butyryl-myo-inosityl)trisphosphate (Scheme 3, Compound 11)

To compound 10 (Scheme 3) (160 mg, 0.13 mmol, 1.0 eq) in EtOH:H₂O (1:1, 6 mL) was added 10% Pd on charcoal (96 mg) and hydrogenated at room temperature for 5 h. The solution was filtered through a LCR/PTFE hydrophilic membrane filter, washed with EtOH:H₂O (1:1, 10 mL) and the combined filtrate was concentrated. The residue was redissolved in water and neutralized with 0.1N NaOH solution. The solvent was concentrated and dried under high vacuum afforded hexasodium 1,3,5-(2,4,6-tri-O-butyryl-myo-inosityl)trisphosphate (Scheme 3, Compound 11) (102 mg, 98%). ¹H NMR (D₂O, 400 MHz, 25° C.): δ=5.83 (bs, 1 H), 5.29 (t, J=9.8 Hz, 2 H), 4.25 (q, J=9.4 Hz, 1 H), 4.19 (t, J=9.7 Hz, 2 H), 2.55 (t, J=7.4 Hz, 2 H), 2.54 (t, J=7.4 Hz, 4 H), 1.78-1.68 (m, 2 H), 1.66-1.57 (m, 4 H), 1.01 (t, J=7.4 Hz, 3 H), 0.94 (t, J=7.4 Hz, 6 H); ³¹P NMR (D₂O, 162 MHz, 25° C.): δ=3.68, 2.79.

### Orthoformate of myo-inositol 2,4,6-tris(dibenzyl phosphate) (Scheme 4, Compound 8)

To a solution of myo-inositol monoorthoformate³² (400 mg, 2.1 mmol, 1 eq) in DCM (5 mL), tetrazole in CH₃CN (0.45 M, 21.0 mL, 9.47 mmol, 4.5 eq) was added at room temperature followed by dibenzyl N,N-diisopropylphosphoramidite (3.1 mL, 9.47 mmol, 4.5 eq). After being stirred for 24 h, the reaction mixture was cooled to −40° C., m-chloroperbenzoic acid (1.81 g, 10.5 mmol, 5 eq) was added portionwise and stirred from −40° C. to room temperature for 12 h. The reaction mixture was diluted with EtOAc, washed with 1N HCl, saturated NaHCO₃, brine, dried (Na₂SO₄) and concentrated in vacuo. The crude was purified by silica gel chromatography (EtOAc/n-heptane, 10:90 to 80:20) afford 1.85 g (90%) of compound 8 (Scheme 4). TLC (SiO₂): R_{f}=0.25 (EtOAc/n-heptane, 50:50); ¹H NMR (CDCl₃, 400 MHz, 25° C.): δ=7.33-7.25 (m, 30 H), 5.49 (d, J=1.2 Hz, 1 H), 5.10-5.02 (obscured, 2 H), 5.06 (d, ³J_{CH}=8.0 Hz, 4 H), 5.01 (d, ³J_{CH}=8.5 Hz, 8 H), 4.89 (dd, J=7.1, 1.3 Hz, 1 H), 4.43-4.40 (m, 1 H), 4.37 (dd, J=2.5, 1.6 Hz, 2 H); ¹³C NMR (CDCl₃, 100 MHz, 25° C.): δ=135.2 (d, ³J_{CP}=6.9 Hz), 135.1 (d, ³J_{CP}=6.4 Hz), 128.45, 128.40, 128.36, 127.8, 127.7, 102.3, 70.2-70.0 (m), 69.7 (d, J=5.5 Hz), 69.67 (d, J=5.4 Hz), 69.4 (d, J=5.7 Hz), 67.6-67.4 (m), 65.5 (d, J=4.8 Hz); ³¹P NMR (CDCl₃, 121 MHz, 25° C.): δ=−0.63; HRMS (ESI-MS): m/z: calcd for C₄₉H₄₉O₁₅P₃Li: 977.2440 [M+Li]⁺; found: 977.2491.

### Orthoformate of hexasodium myo-inositol 2,4,6-trisphosphate (Scheme 4, Compound 9)

To compound 8 (Scheme 4) (310 mg, 0.31 mmol, 1.0 eq) in EtOH:H₂O (1:1, 10 mL) was added 10% Pd on charcoal (160 mg), NaHCO₃ (161 mg, 1.91 mmol, 6.0 eq) and hydrogenated at room temperature for 12 h. The solution was filtered through a LCR/PTFE hydrophilic membrane filter, washed with EtOH:H₂O (1:1, 20 mL) and the combined filtrate was concentrated and dried under high vacuum afforded 175 mg (97%) of compound 9 (Scheme 4). ¹H NMR (D₂O, 400 MHz, 25° C.): δ=5.62 (s, 1 H), 4.84-4.75 (obscured, 2 H), 4.61 (d, J=9.2 Hz, 1 H), 4.48 (bs, 1 H), 4.43 (bs, 2 H); ¹³C NMR (D₂O, 100 MHz, 25° C.): δ=102.3, 73.0 (t, J=3.1 Hz), 70.0 (t, J=3.9 Hz), 68.4 (d, J=4.3 Hz), 62.6 (d, J=4.1 Hz); ³¹P NMR (D₂O, 162 MHz, 25° C.): δ=4.43, 4.06; HRMS (ESI-MS): m/z: calcd for C₇H₈O₁₅P₃Na₆: 562.8457 [M+H]⁺; found: 562.8488.

### scyllo-inositol hexakis(dibenzyl phosphate) (Scheme 5, Compound 1)

To a solution of scyllo-inositol (360 mg, 2 mmol, 1 eq) in DMF (40 mL) tetrazole in CH₃CN (0.45 M, 53.3 mL, 24 mmol, 12 eq) was added at room temperature followed by dibenzyl N,N-diisopropylphosphoramidite (5.9 mL, 18 mmol, 9 eq). After being stirred for 24 h, the reaction mixture was cooled to 0° C. Then sodium phosphate buffer (1 N, pH=7, 50 mL) was added followed by 30% H₂O₂ (50 mL) and stirred from 0° C. to room temperature for 12 h. The reaction mixture was diluted with EtOAc and the aqueous phase was separated. The organic layer was washed with 1N HCl, saturated NaHCO₃, brine, dried (Na₂SO₄) and concentrated in vacuo. The residue was purified by silica gel column chromatography (EtOAc/n-heptane, 10:90 to 80:20) to obtain 1.94 g (55%) of scyllo-inositol hexakis(dibenzyl phoshphate) (Scheme 5, Compound 1) as light yellow oil. TLC (SiO₂): R_{f}=0.25 (EtOAc/n-heptane, 50:50); ¹H NMR (CDCl₃, 400 MHz, 25° C.): δ=7.22-7.18 (m, 60 H), 5.18 (d, ³J_{HP}=7.4 Hz, 6 H), 5.07-4.95 (m, 24 H); ¹³C NMR (CDCl₃, 100 MHz, 25° C.): δ=135.6 (d, ³J_{CP}=7.0 Hz), 128.37, 128.27, 128.02, 76.6 (d, J=7.7 Hz), 69.9 (d, ²J_{CP}=5.6 Hz); ³¹P NMR (CDCl₃, 121 MHz, 25° C.): δ=−0.73; HRMS (ESI-MS): m/z: calcd for C₉₀H₉₀O₂₄P₆NaLi: 885.2148 [M+Na+Li]⁺²; found: 885.2293.

### Hexatriethylammonium scyllo-inositol hexakisphosphate (Scheme 5, Compound 2)

To scyllo-inositol hexakis(dibenzyl phoshphate) (Scheme 5, Compound 1) (870 mg, 0.50 mmol, 1.0 eq) in EtOH:H₂O (1:1, 50 mL) was added 10% Pd on charcoal (500 mg) and hydrogenated at room temperature for 12 h. The solution was filtered through a LCR/PTFE hydrophilic membrane filter, washed with EtOH:H₂O (1:1, 40 mL) and the combined filtrate was evaporated and dried under high vacuum afforded debenzylated product. This product (323 mg, 0.49 mmol, 1.0 eq) was dissolved in H₂O (5 mL) and Et₃N (1.63 mL, 11.76 mmol, 24 eq) was added at room temperature and stirred for 30 minutes. Then the solvent was concentrated and dried under high vacuum to get 607 mg (98% for two steps) of hexatriethylammonium scyllo-inositol hexakisphoshphate (Scheme 5, Compound 2). ¹H NMR (D₂O, 400 MHz, 25° C.): δ=4.20 (d, ³J_{HP}=4.8 Hz, 6 H), 3.16 (q, J=7.3 Hz, 36 H), 1.23 (t, J=7.3 Hz, 54 H); ¹³C NMR (D₂O, 100 MHz, 25° C.): δ=76.6 (bs), 46.8, 8.4; ³¹P NMR (D₂O, 121 MHz, 25° C.): δ=1.67.

### Hexatriethylammonium scyllo-inositol 1,2:3,4:5,6-trispyrophosphate (Scheme 5, Compound 3)

To a solution of hexatriethylammonium scyllo-inositol hexakisphoshphate (Scheme 5, Compound 2) (607 mg, 0.48 mmol, 1 eq) in H₂O (3 mL) 1,3-dicyclohexylcarbodiimide (594 mg, 2.87 mmol, 6 eq) in CH₃CN was added (6 mL) and refluxed for 6 h. Two more equivalents of 1,3-dicyclohexylcarbodiimide (99 mg, 0.48 mmol, 1 eq) was added at 4 h intervals and refluxed for further 8 h. The reaction mixture was diluted with water (5 mL), dicyclohexylurea was filtered through a sintered funnel and washed with water (2×10 mL). The combined filtrate was evaporated on a rotary evaporator (55° C.) and dried under high vacuum. The resulting residue was redissolved in 20 mL of water and filtered through a sintered funnel, washed with water (2×5 mL) to remove any further amount of dicyclohexylurea that was dissolved in acetonitrile. The combined filtrate was evaporated on a rotary evaporator (55° C.) and dried under high vacuum afforded hexatriethylammonium scyllo-inositol 1,2:3,4:5,6-trispyrophosphate (Scheme 5, Compound 3). ¹H NMR (D₂O, 400 MHz, 25° C.): δ=4.41 (bs, 6 H), 3.20 (q, J=7.3 Hz, 36 H), 1.28 (t, J=7.3 Hz, 54 H); ¹³C NMR (D₂O, 100 MHz, 25° C.): δ=76.2 (bs), 46.8, 8.4; ³¹P NMR (D₂O, 121 MHz, 25° C.): δ=−10.10.

### Hexasodium scyllo-inositol 1,2:3,4:5,6-trispyrophosphate (Scheme 5, Compound 4)

Hexatriethylammonium scyllo-inositol 1,2:3,4:5,6-trispyrophosphate (Scheme 5, Compound 3) was dissolved in water (10 mL) and treated with Dowex Na⁺ form (10 g) for 1 h. The solution was filtered, washed with water (2×5 mL). To the filtrate fresh Dowex Na⁺ form (10 g) was added, stirred for 1 h and filtered. This process was repeated until all the triethyl ammonium ions are exchanged with sodium ions. Finally the solvent was evaporated under reduced pressure and dried under high vacuum to yield hexasodium scyllo-inositol 1,2:3,4:5,6-trispyrophosphate 4, (Scheme 5, 339 mg, 96%) along with small amount of pyrophosphate hydrolyzed product. ¹H NMR (D₂O, 400 MHz, 25° C.): δ=4.44 (s, 6 H); ¹³C NMR (D₂O, 100 MHz, 25° C.): δ=76.2 (s); ³¹P NMR (D₂O, 121 MHz, 25° C.): δ=−9.92; HRMS (ESI-MS): m/z: calcd for C₆H₆O₂₁P₆Na₇: 760.7106 [M+Na]⁺; found: 760.7142.

### Hydrolysis and Alcoholysis of myo-inositol 1,6:2,3:4:5-trispyrophosphate hexasodium salt (Scheme 6)

A solution of myo-inositol 1,6:2,3:4:5-trispyrophosphate hexasodium salt (400 mg, 0.54 mmol, 1 eq) in water (5 mL) was passed through a Dowex 50WX8-200 (10 g) column and the column was washed with water (4×5 mL). Alternatively, the hydrolysis can be achieved by dissolving the trispyrophosphate hexasodium salt in 1 normal HCl solution. The acidic fractions were pooled and stirred at room temperature for 24 h. Then the pH of the solution was adjusted around 7 with 0.1N NaOH solution. Then the solvent was evaporated to dryness to get a mixture of partial pyrophosphate hydrolyzed product 5 (Scheme 6, 424 mg) as a white solid. ¹H NMR (D₂O, 400 MHz, 25° C.): δ=4.99-4.88 (d, J=9.8 Hz, global integration 1 H), 4.62-4.35 (m, global integration 5 H); ³¹P NMR (D₂O, 162 MHz, 25° C.): δ=0.41, 0.17, 0.07, −0.24, −0.31, −0.45, −0.90, −1.12, −1.28, −1.34, −1.42 (singlet's, global integration 2.7 P), −10.81 & −11.16 to −11.42 (AB and multiplet, ²J_{PP}=17.5 Hz, global integration 3.3 P); HRMS (ESI-MS): m/z: calcd for C₆H₇O₂₂P₆Na₈: 800.7031 [M+H]⁺; found: 800.7031

To a solution of myo-inositol 1,6:2,3:4:5-trispyrophosphate hexasodium salt (300 mg, 0.4 mmol, 1 eq) in dry MeOH (4 mL), acetyl chloride (1.0 mL, 14.0 mmol, 35 eq) was added at 0° C. and stirred from 0° C. to room temperature for 4 h. Then the solution was evaporated under reduced pressure and dried under high vacuum. The resulting residue was dissolved in water and adjusted the pH around 7 with 0.1N NaOH solution. Then the solvent was concentrated and dried under high vacuum afforded a mixture of pyrophosphate opened product 6 (Scheme 6, 365 mg) as a white solid. ¹H NMR (D₂O, 400 MHz, 25° C.): δ=5.05 & 4.97 & 4.89-4.86 (doublets and multiplet, J=9.2 Hz, J=8.8 Hz, global integration 1 H), 4.56-4.45 (m, global integration 2 H), 4.25-4.08 (m, global integration 3 H), 3.73-3.64 (m, global integration 9 H); ³¹P NMR (D₂O, 162 MHz, 25° C.): δ=2.24, 2.11, 1.83, 1.69, 1.50, 1.45, 1.32, 1.17, 1.10, 1.01, 0.89, 0.63, 0.44, 0.01, −0.46, (singlet's, global integration 6 P); HRMS (ESI-MS): m/z: calcd for C₉H₁₅O₂₄P₆Na₁₀: 922.7350 [M+Na]⁺; found: 922.7408.

To a solution of myo-inositol 1,6:2,3:4:5-trispyrophosphate hexasodium salt (300 mg, 0.4 mmol, 1 eq) in dry MeOH (4 mL), acetyl chloride (0.1 mL, 1.4 mmol, 3.5 eq) was added at 0° C. and stirred from 0° C. to room temperature for 36 h. Then the solution was concentrated in vacuo and the resulting residue was dissolved in water and adjusted the pH around 7 with 0.1N NaOH solution. Then the solvent was evaporated and dried under high vacuum to get the a mixture of partial pyrophosphate opened product 7 (Scheme 6, 321 mg) along with small amount of starting material. ¹H NMR (D₂O, 400 MHz, 25° C.): δ=5.19-4.87 (m, global integration 1 H), 4.67-4.13 (m, global integration 5 H), 3.78-3.67 (m, global integration 3 H); ³¹P NMR (D₂O, 162 MHz, 25° C.): δ=2.36 to −1.11 (many singlet's, global integration 3.5 P), −9.38, −10.04 to −11.51 & −14.18 (AB and multiplet, ²J_{PP}=21.6 Hz, global integration 2.5 P); HRMS (ESI-MS): m/z: calcd for C₇H₉O₂₂P₆Na₈: 814.7187 [M+H]⁺; found: 814.7201.

### EXAMPLE 1

### In Vitro Experiments Performed on Free Hemoglobin and On Whole Human Blood

Some of the compounds described herein were tested for P₅₀ on free hemoglobin (Hb) as well as human whole blood (WB) as 120 mM solutions. The hemoglobin solution was prepared from red blood cells concentrate (EFS-Alsace) by washing three times with 1 volume of saline (1500×g, 10 min), the cells were hemolysed by addition of 2 volumes of cold distilled water. After centrifugation (7000×g, 30 min) for stroma removal, 5 ml of the clear hemoglobin solution were placed on a 2.5 cm×30 cm column of Sephadex G-25 equilibrated with 0.1 M sodium chloride+10⁻⁵ M EDTA. The protein was eluted with the same solution at a rate of about 20 ml/h [Benesch, R.; Benesch, R. E. and Yu, C. I. Reciprocal binding of oxygen and diphosphoglycerate by human hemoglobin. Proc. Natl. Acad. Sci. USA (1968) 59, 526-532].

The allosteric modulation of the effectors was measured by the change in p50, the partial pressure of oxygen for half-saturation. myo-Inositol hexaphosphate (myo-IHP) was purchased from Sigma. Oxygen equilibrium curves (OEC) were carried out with the Hemox Analyzer (TCS Scientific Co.) under the following conditions: pH 7.4, 135 mM NaCl, 5 mM KCl and 30 mM TES (N-[tris(hydroxymethyl)methyl]-2-aminoethanesulfonic acid) buffer at 37° C. The concentration of free hemoglobin was 100 μM (577 nm, ε=58.4 mM⁻¹cm⁻¹ per tetramer) and the final concentration of the allosteric effector in the cuvette was 2 mM resulting in an effector/Hb ratio of 20.

Human blood was freshly withdrawn in heparinized tubes. The pH of the compound solutions was adjusted to approximately 7.0 and whole blood volumes at 1:1 ratios where incubated individually for two hours at 37° C. with the above compounds. Following, incubation, blood cells were washed 3 times with Bis-Tris-buffer. The measurement of oxygen dissociation curves was made in a Hemox-Analyzer instrument (TCS Scientific Corp.) A summary of P₅₀ values for whole blood induced by the compounds is presented in Table 1.

TABLE 1

| | | | | | P50 | |
|---|---|---|---|---|---|---|
| | Blood | | P50 control | P50 | increase | |
| Compound | matrix | n | (Torr) | (Torr) | (%) | Structure |
| myo-IHP(reference) | Hb | 3 | 12.66 ±1.62 | 48.37 ±3.71 | 282 | 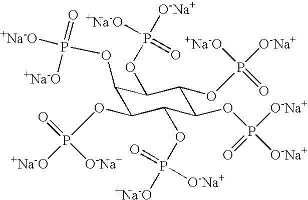 |
| myo-IHP.3 Me(Compound 6,Scheme 6) | Hb | 3 | 10.72 ±0.37 | 37.56 ±1.30 | 250 | 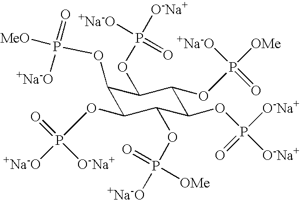 |
| scyllo-IHPsodium salt | Hb | 3 | 12.20 ±0.27 | 36.37 ±1.55 | 198 | 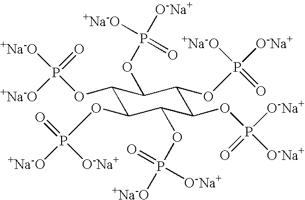 |
| scyllo-ITPP(Compound 4,Scheme 5) | HbWB | 33 | 10.14 ±0.06 | 23.02 ±1.8334.10 ±1.81 | 127 18 | 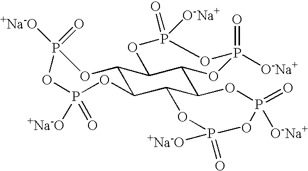 |
| myo-Inositol(Compound 7,Scheme 6) | Hb | 3 | 10.62 ±0.19 | 27.43 ±1.07 | 158 | 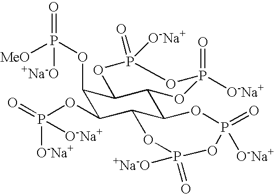 |
| myo-Inositol(Compound 5,Scheme 6) | HbWB | 33 | 10.98 ±0.7728.82 ±0.73 | 27.25 ±0.1435.53 ±1.43 | 148 23 | 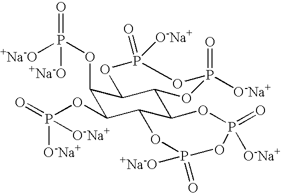 |

### REFERENCES

1. Marshall et al. J. Exp. Biol. 1993, 184:161-182.
2. Wilcox et al. Biochem J. 1993, 294:191-194.
3. Morris et al. Nature, 1987, 330:653-655.
4. Putney et al. FASEB J. 1989, 3:1899-1905.
5. Ely et al. Biochem. J. 1990, 268:333-338.
6. Guse et al. Biochem. J. 1992, 288:489-495.
7. Luckhoff et al. Nature, 1992, 355:356-358.
8. Irvine, R. T. in Advances in Second Messenger and Phosphoprotein Research (Vol 26) (Putney, J. W. Ed.) pp 161-185 Raven Press, New York, N.Y.
9. Mayr, G. W. Biochem J. 1989, 259:463-470.
10. Kuo, T. H. Biochem. Biophys. Res. Commun. 1988, 152:1111-1118.
11. Davis et al. FASEB J. 1991, 5:2992-2995.
12. Ivorra et al. Biochem J. 1991, 273:317-321.
13. Gawler et al. Biochem J. 1991, 273:161-167.
14. Good et al. PNAS, 1990, 87:6624-6628.
15. O'Reilly et al. Cell, 1994, 79:315-328.
16. Chen et al. Cancer Res. 1995, 55:4230-4233.
17. Ferrara, N. Nat. Rev. Cancer 2002, 2:795-803.
18. Ferrara et al. Endocr. Rev. 1997, 18:4-25.
19. Ferrara et al. Nat. Med. 2003, 9:669-676.
20. Fontanini et al. Clin. Cancer Res. 1997, 3:861-865.
21. Brizel et al. Cancer Res. 1996, 56:941-943.
22. Ishii et al. J. Nucl. Med. 1996, 37:1159-1165.
23. Hirst et al. Radiat. Res. 1987, 112:164.
24. Nahorski, S. British J. of Pharmacology, 2006, 147:S38-S45.
25. Lu et al. Biochemistry, 1994, 33:11586-11597.
26. Irvine et al. Nat. Rev. Mol. Cell. Biol. 2001, 2:327-358.
27. Saiardi et al. Science, 2004, 306:2101-2105.
28. Challis et al. Biochem. Biophys. Res. Comm. 1988, 157:684-691.
29. Smith, P. Biochem J. 1992, 283:27-30
30. Dor et al. Am. J. Physiol. Cell Pysiol. 2001, 280:C1367-1374.
31. Dinkel, C.; Moody, M.; Traynor-Kaplan, A.; Schultz, C. Angew. Chem. Int. Ed. 2001, 40, 3004-3008.
32. Lee, H. W.; Kishi, Y. J. Org. Chem. 1985, 50, 4402-4404.

We claim:

1. A hexakisphosphate inositol derivative wherein the inositol is cis-inositol, epi-inositol, allo-inositol, muco-inositol, neo-inositol, scyllo-inositol, (+) chiro-inositol, or (−) chiro-inositol.

2. The hexakisphosphate inositol derivative of claim 1, wherein the inositol derivative is complexed with a cation to form a salt, and wherein the cation is a alkali metal cation, an alkaline metal cation, an ammonium cation, or an organic cation.

3. A pharmaceutical composition comprising the hexakisphophate inositol derivative of claim 2.

4. A polyphosphorylated inositol derivate wherein the inositol comprises one or more free hydroxyl or hydroxyl derivative groups.

5. The inositol derivative of claim 4, wherein the hydroxyl derivative is an alkoxy (—OR) or an acyloxy (—OCOR) derivative.

6. The inositol derivative of claim 5, wherein R is selected from one or more of the following; alkyl, aryl, acyl, aralkyl, alkenyl, alkynyl, heterocyclyl, polycyclyl, carbocycle, amino, acylamino, amido, alkylthio, carbonyl, sulfonate, alkoxyl, sulfonyl, or sulfoxido.

7. The inositol derivative of claim 5, wherein the inositol derivative is complexed with a cation to form a salt, and wherein the cation is a alkali metal cation, an alkaline metal cation, an ammonium cation, or an organic cation.

8. A pharmaceutical composition comprising the inositol derivative of claim 7.

9. A pyrophosphate inositol derivative, wherein the inositol is cis-inositol, epi-inositol, allo-inositol, muco-inositol, neo-inositol, scyllo-inositol, (+) chiro-inositol, or (−) chiro-inositol, and wherein the inositol derivative is a monpryophosphate, a bispyrophosphate, or a trispyrophosphate derivative.

10. The pyrophosphate inositol derivative of claim 9, wherein the inositol derivative is complexed with a cation to form a salt, and wherein the cation is an alkali metal cation, an alkaline metal cation, an ammonium cation, or an organic cation.

11. A pharmaceutical composition comprising the pyrophosphate derivative of claim 10.

12. A pyrophosphate myo-inositol derivative, wherein the myo-inositol comprises a bispyrophosphate.

13. The pyrophosphate myo-inositol derivative of claim 12, wherein the myo-inositol derivative is complexed with a cation to form a salt, and wherein the cation is an alkali metal cation, an alkaline metal cation, an ammonium cation, or an organic cation.

14. A pharmaceutical composition comprising the pyrophosphate myo-inositol derivative of claim 13.

15. A thiophosphorimide inositol derivative.

16. The trisphosphorimide inositol derivative of claim 15, wherein the inositol derivative is complexed with a cation to form a salt, and wherein the cation is an alkali metal cation, an alkaline metal cation, an ammonium cation, or an organic cation.

17. A pharmaceutical composition comprising the trisphosphorimide inositol derivative of claim 16.

18. A tristhiopyrophosphate inositol derivative.

19. The tristhiopyrophosphate inositol derivative of claim 18, wherein the inositol derivative is complexed with a cation to form a salt, and wherein the cation is an alkali metal cation, an alkaline metal cation, an ammonium cation, or an organic cation.

20. A pharmaceutical composition comprising the tristhiopyrophosphate derivate of claim 19.

21. A method of reducing the affinity of hemoglobin for red blood cells in a human or animal comprising administering to the human or animal an effective amount of a polyphosphate derivative or pyrophosphate derivative of inositol.

22. The method of claim 21, wherein the polyphosphate derivative is a hexakisphosphate derivative of cis-inositol, epi-inositol, allo-inositol, muco-inositol, neo-inositol, scyllo-inositol, (+) chiro-inositol, or (−) chiro-inositol.

23. The method of claim 21, wherein the polyphosphate derivative comprises one or more free hydroxyl or hydroxyl derivative groups.

24. The method of claim 21, wherein the pyrophosphate derivative is a trispyrophosphate of cis-inositol, epi-inositol, allo-inositol, muco-inositol, neo-inositol, scyllo-inositol, (+) chiro-inositol, or (−) chiro-inositol.

25. The method of claim 21, wherein the pyrophosphate derivative is a myo-inositol bisphosphate.

26. The method of claim 21, wherein the pyrophosphate derivative is a trisphosphorimide.

27. The method of claim 21, wherein the pyrophosphate derivate is a tristhiopyrophosphate derivative.

28. A method of treating an ischemia mediated disease comprising administering to a patient with an ischemic disease a therapeutically effective amount of the hexakisphosphate inositol derivative of claim 1.

29. The method of claim 28, where in the ischemia mediated disease is Alzheimer's disease, dementia, stroke, chronic obstructive pulmonary disease (COPD), osteoporosis, or adult respiratory distress syndrome (ARDS).

30. A method of treating an angiogenesis mediated disease comprising administering to a patient with an ischemic disease a therapeutically effective amount of the hexakisphosphate inositol derivative of claim 1.

31. The method of claim 30, wherein the angiogenesis mediated disease is excessive or abnormal stimulation of endothelial cells (e.g. atherosclerosis), blood borne tumors, solid tumors and tumor metastasis, benign tumors, for example, hemangiomas, acoustic neuromas, neurofribromas, trachomas, and pyogenic granulomas, vascular malfunctions, abnormal wound healing, inflammatory and immune disoreders, Bechet's disease, gout, or gouty arthritis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease), breast cancer, prostate cancer, renal cell cancer, brain cancer, ovarian cancer, colon cancer, bladder cancer, pancreatic cancer, stomach cancer, esophageal cancer, cutaneous melanoma, liver cancer, lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip and oral cancer, skin cancer, leukemia or multiple myeloma.
